(19) United States
(12) Patent Application Publication
(10) Pub. No.: US 2024/0081667 A1
(43) Pub. Date: Mar. 14, 2024

(54) ELECTRONIC SPHYGMOMANOMETER AND BLOOD PRESSURE MEASUREMENT METHOD

(71) Applicant: OMRON HEALTHCARE CO., LTD., Muko-shi (JP)

(72) Inventors: Naomi MATSUMURA, Kyoto (JP); Yukiya SAWANOI, Kyoto (JP)

(73) Assignee: OMRON HEALTHCARE CO., LTD., Muko-shi (JP)

(21) Appl. No.: 18/511,175

(22) Filed: Nov. 16, 2023

Related U.S. Application Data
(63) Continuation of application No. PCT/JP22/26608, filed on Jul. 4, 2022.

(30) Foreign Application Priority Data
Jul. 29, 2021 (JP) 2021-124560

(51) Int. Cl. A61B 5/022; A61B 5/00; A61B 5/021

(52) U.S. Cl. CPC A61B 5/02233; A61B 5/02116; A61B 5/7235; A61B 2560/02; A61B 2560/0462; A61B 2562/0247

(57) ABSTRACT

The present invention includes a pressing cuff for compressing a measurement target site. A sensing cuff containing a pressure transmission fluid is provided on an inner circumferential side of the pressing cuff. A pressure at a rising start point and a pressure at a peak point indicated by a pressure pulse wave for each beat are obtained by subtracting a direct current component of data representing a pressure of the pressing cuff from data representing a pressure of the sensing cuff in the process of depressurization. A first time point at which the pressure at the peak point for each beat transitions between a zero level and a positive value is obtained. A second time point at which the pressure at the rising start point for each beat transitions between the zero level and the positive value is obtained.

### CROSS-REFERENCE TO RELATED APPLICATIONS

This is a continuation application of International Application No. PCT/JP2022/026608, with an International filing date of Jul. 4, 2022, which claims priority of Japanese Patent Application No. 2021-124560 filed on Jul. 29, 2021, the entire content of which is hereby incorporated by reference.

### TECHNICAL FIELD

The present invention relates to an electronic sphygmomanometer, and more specifically to an electronic sphygmomanometer and a blood pressure measurement method for noninvasively measuring blood pressure at a measurement target site.

### BACKGROUND ART

Conventionally, as this type of electronic sphygmomanometer, there is known an electronic sphygmomanometer that compresses a measurement target site with a blood pressure cuff and calculates blood pressure of the measurement target site by using an oscillometric method as disclosed in Patent Document 1 (JP-H09-299339 A), for example. Specifically, when the blood pressure cuff is in a pressurization process or a depressurization process, an envelope is set for a row of pulse wave amplitudes obtained from the cuff pressure, threshold levels (including a threshold level for systole and a threshold level for diastole) of a predetermined proportion (ratio) are set with respect to a maximum value of the envelope, and the cuff pressure at a time point when the envelope crosses these threshold levels are calculated as a maximum blood pressure (systolic blood pressure) and a minimum blood pressure (diastolic blood pressure), respectively.

### SUMMARY OF THE INVENTION

For example, the threshold level for systolic blood pressure is set to 0.5 to 0.6, and the threshold level for diastolic blood pressure is set to 0.7 to 0.8. This is to statistically match the systolic blood pressure and the diastolic blood pressure calculated by the oscillometric method with the systolic blood pressure and the diastolic blood pressure measured by a reference measurement method (for example, a traditional auscultatory method), respectively.

For this reason, although accuracy is statistically secured for the systolic blood pressure and the diastolic blood pressure calculated by the oscillometric method, there is a problem that there is a person who has a difference from the systolic blood pressure and the diastolic blood pressure measured by the reference measurement method.

An object of the present invention is to provide an electronic sphygmomanometer and a blood pressure measurement method capable of correctly measuring blood pressure at a measurement target site in principle in a noninvasive manner. Here, “capable of correctly measuring blood pressure in principle” means that the measurement is based on the same mechanism as the principle of the measurement method as a reference.

In order to achieve the object, an electronic sphygmomanometer of the present disclosure is an electronic sphygmomanometer that noninvasively measures blood pressure at a measurement target site, the electronic sphygmomanometer comprising:
a pressing cuff having a bag shape configured to be attached around the measurement target site along a circumferential direction of the measurement target site to receive supply of a pressurization fluid and compress the measurement target site;
a sensing cuff having a bag shape disposed in a portion facing an artery of the measurement target site on an inner circumferential side of the pressing cuff, the sensing cuff containing a pressure transmission fluid separately from the pressing cuff, the sensing cuff configured to compress the artery of the measurement target site via the pressure transmission fluid by using a pressure of the pressing cuff and receive a pressure pulse wave from the artery;
a first pressure sensor that detects a pressure of the sensing cuff;
a second pressure sensor that detects the pressure of the pressing cuff;
a pressure control unit that controls the pressure of the pressing cuff by supplying the pressurization fluid to the pressing cuff or discharging the pressurization fluid from the pressing cuff; and
a blood pressure calculation unit that calculates a blood pressure value based on data representing the pressure of the sensing cuff from the first pressure sensor and data representing the pressure of the pressing cuff from the second pressure sensor in a process of changing the pressure of the pressing cuff with the pressure control unit,
wherein the blood pressure calculation unit
obtains a pressure at a rising start point and a pressure at a peak point indicated by the pressure pulse wave for each beat by subtracting a direct current component of data representing the pressure of the pressing cuff or a value approximate to the direct current component from data representing the pressure of the sensing cuff in the process of changing the pressure; and
obtains a first time point at which the pressure at the peak point for each beat transitions between a zero level and a positive value in the process of changing the pressure, obtains the pressure of the pressing cuff at the first time point as a systolic blood pressure value, obtains a second time point at which the pressure at the rising start point for each beat transitions between the zero level and the positive value, and obtains the pressure of the pressing cuff at the second time point as a diastolic blood pressure value.

In the present specification, the “pressure transmission fluid” may be enclosed in the sensing cuff at the manufacturing stage of the electronic sphygmomanometer, or may be contained in the sensing cuff and discharged from the sensing cuff every time the blood pressure is measured.

The “fluid” for pressurization fluid and pressure transmission fluid is typically air, but it may be another gas or liquid.

The “direct current component” of the data representing the pressure of the pressing cuff means a component obtained by removing a fluctuation component (for example, a fluctuation component for each beat derived from a pressure pulse wave from the artery) from the data representing the pressure of the pressing cuff. The “value approximate to the direct current component” refers to, for example, in a depressurization process at a constant depressurization speed, a value approximate to a straight line determined by data of one point of the pressure of the pressing cuff and the depressurization speed.

The “obtains a pressure at a rising start point and a pressure at a peak point indicated by the pressure pulse wave for each beat” means to obtain the pressure at the rising start point and the pressure at the peak point when the pressure pulse wave detected by the first pressure sensor indicates the rising start point and the peak point for each beat. For example, in a pressure section in which the pressure of the pressing cuff is higher than the systolic blood pressure value in the depressurization process, the blood flow in the artery is stopped, and thus, the “rising start point” and the “peak point” are not observed, and therefore, these pressures are not obtained.

The “first time point” and the “second time point” are names for convenience of distinguishing these time points from each other, and do not necessarily mean the order of these time points.

In another aspect, a blood pressure measurement method of the present disclosure is a blood pressure measurement method for noninvasively measuring blood pressure at a measurement target site, wherein
a pressing cuff having a bag shape configured to be attached around the measurement target site along a circumferential direction of the measurement target site to receive supply of a pressurization fluid and compress the measurement target site;
a sensing cuff having a bag shape disposed in a portion facing an artery of the measurement target site on an inner circumferential side of the pressing cuff, the sensing cuff containing a pressure transmission fluid separately from the pressing cuff, the sensing cuff configured to compress the artery of the measurement target site via the pressure transmission fluid by using a pressure of the pressing cuff and receive a pressure pulse wave from the artery;
a first pressure sensor that detects a pressure of the sensing cuff;
a second pressure sensor that detects the pressure of the pressing cuff; and
a pressure control unit that controls the pressure of the pressing cuff by supplying the pressurization fluid to the pressing cuff or discharging the pressurization fluid from the pressing cuff are provided,
the blood pressure measurement method comprising:
acquiring data representing the pressure of the sensing cuff from the first pressure sensor and data representing the pressure of the pressing cuff from the second pressure sensor in a process of changing the pressure of the pressing cuff with the pressure control unit;
obtaining a pressure at a rising start point and a pressure at a peak point indicated by the pressure pulse wave for each beat by subtracting a direct current component of data representing the pressure of the pressing cuff or a value approximate to the direct current component from data representing the pressure of the sensing cuff in the process of changing the pressure; and
obtaining a first time point at which the pressure at the peak point for each beat transitions between a zero level and a positive value in the process of changing the pressure, obtaining the pressure of the pressing cuff at the first time point as a systolic blood pressure value, obtaining a second time point at which the pressure at the rising start point for each beat transitions between the zero level and the positive value, and obtaining the pressure of the pressing cuff at the second time point as a diastolic blood pressure value.

### BRIEF DESCRIPTION OF THE DRAWINGS

FIG. 1 is a diagram showing a block configuration of an electronic sphygmomanometer (hereinafter, it is abbreviated as a “sphygmomanometer”) according to an embodiment of the present invention.

FIG. 2 is a diagram showing a section of a cuff (including a pressing cuff and a sensing cuff) constituting the sphygmomanometer in FIG. 1 attached to a measurement target site together with an air pipe system.

FIG. 3 is a diagram showing a schematic flow of blood pressure measurement by the sphygmomanometer in FIG. 1.

FIG. 4 is a diagram showing a detailed flow of how to calculate blood pressure included in the flow of FIG. 3.

FIG. 5A is a diagram showing data representing a pressure Pc of the pressing cuff and data representing a pressure Ps of the sensing cuff in a depressurization process.

FIG. 5B is a diagram showing data representing a waveform (pressure) of a pressure pulse wave PW obtained by subtracting a direct current component of the data representing the pressure Pc of the pressing cuff from the data representing the pressure Ps of the sensing cuff with the cuff pressure Pc on the horizontal axis.

FIG. 6 is an enlarged view of a vicinity of a portion of FIG. 5B where the cuff pressure Pc on the horizontal axis corresponds to a diastolic blood pressure value DIA.

FIG. 7 is a diagram for illustrating in principle how to calculate the blood pressure with the sphygmomanometer.

FIG. 8 is a diagram showing a correspondence relationship (tube law) between an internal and external pressure difference of an artery and a volume of the artery.

FIGS. 9A to 9E are diagrams showing various arrangements that may be taken by a first air pipe connected to the sensing cuff.

FIG. 10A is a sectional view showing another arrangement that may be taken by the first air pipe.

FIG. 10B is a plan view showing the arrangement that may be taken by the first air pipe.

FIG. 10C is a sectional view of the cuff including an inner cloth.

FIG. 11 is a diagram showing a block configuration of a sphygmomanometer according to a modification of the sphygmomanometer in FIG. 1.

FIG. 12A is a diagram showing a section of a cuff constituting the sphygmomanometer in FIG. 11 attached to a measurement target site together with an air pipe system.

FIG. 12B is a diagram showing a state in which a switching valve included in the air pipe system is switched to a second position (operation position).

FIG. 13 is a diagram showing a schematic flow of blood pressure measurement by the sphygmomanometer in FIG. 11.

FIG. 14 is a diagram showing another schematic flow of blood pressure measurement by the sphygmomanometer in FIG. 1.

FIG. 15 is a diagram showing a detailed flow of how to calculate blood pressure included in the flow of FIG. 14.

FIG. 16A is a diagram showing data representing a pressure Pc of the pressing cuff and data representing a pressure Ps of the sensing cuff in a pressurization process.

FIG. 16B is a diagram showing data representing a waveform (pressure) of a pressure pulse wave PW obtained by subtracting a direct current component of the data representing the pressure Pc of the pressing cuff from the data representing the pressure Ps of the sensing cuff with the cuff pressure Pc put on the horizontal axis.

FIG. 17A is a diagram showing a state in which a general cuff compresses a measurement target site.

FIG. 17B is a diagram showing data representing a pressure Pc′ of the general cuff and data of a waveform (pressure) of a pressure pulse wave PW′ included in the pressure Pc′ in a depressurization process.

### DETAILED DESCRIPTION

Hereinafter, embodiments of the present invention will be described in detail with reference to the drawings.

(Configuration of Sphygmomanometer)

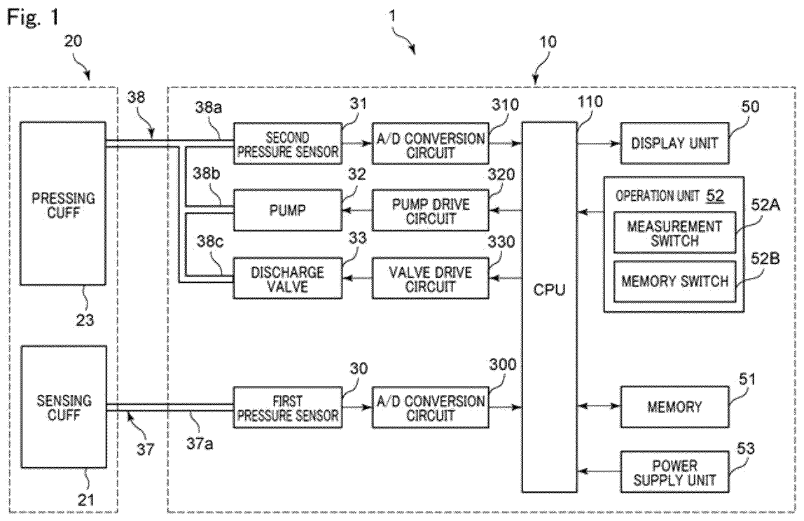

FIG. 1 shows a schematic block configuration of a sphygmomanometer 1 according to an embodiment of the present invention. The sphygmomanometer 1 mainly includes a cuff 20 configured to be attached to a measurement target site such as a wrist or an upper arm (in this example, an upper arm), and a main body 10.

The cuff 20 includes a bag-shaped pressing cuff 23 for receiving supply of air as a pressurization fluid to compress a measurement target site 90, and a bag-shaped sensing cuff 21 for containing air as a pressure transmission fluid separately from the pressing cuff 23.

The main body 10 is mounted with a central processing unit (CPU) 110 as a control unit, a display 50, a memory 51 as a storage unit, an operation unit 52, a power supply unit 53, a first pressure sensor 30, a second pressure sensor 31, a pump 32, and a discharge valve 33. The main body 10 further includes A/D conversion circuits 300 and 310 for converting an analog signal into a digital signal, a pump drive circuit 320 for driving the pump 32, and a valve drive circuit 330 for driving the discharge valve 33. The air pipe 37a connected to the first pressure sensor 30 is connected to the sensing cuff 21 in a fluid communicable manner as a flexible first air pipe 37 (in this example, the air pipe 37a is collectively referred to as the first air pipe 37). Air pipes 38a, 38b, and 38c respectively connected to the second pressure sensor 31, the pump 32, and the discharge valve 33 are joined into one pipe as a flexible second air pipe 38, and are connected to the pressing cuff 23 in a fluid communicable manner (in this example, the air pipes 38a, 38b, and 38c are collectively referred to as the second air pipe 38). In this example, the first air pipe 37 and the second air pipe 38 constitute a first fluid pipe and a second fluid pipe, respectively. In this example, the first air pipe 37 and the second air pipe 38 are completely separated from each other. Thus, for example, it is possible to prevent mixing of a fluctuation component (caused by a pressure pulse wave from the artery, for example) included in the pressure of the first air pipe 37 connected to the sensing cuff 21 as noise into the pressure of the second air pipe 38 connected to the pressing cuff 23. Conversely, it is possible to prevent mixing of a fluctuation component (mainly caused by vibration of the pump 32) included in the pressure of the second air pipe 38 connected to the pressing cuff 23 as noise into the pressure of the first air pipe 37 connected to the sensing cuff 21. Hereinafter, the first air pipe 37 and the second air pipe 38 are collectively referred to as air pipe systems 37 and 38 as appropriate.

The display 50 includes a display and an indicator, and displays predetermined information (for example, a blood pressure measurement result or the like) according to a control signal from the CPU 110.

In this example, the operation unit 52 includes a measurement switch 52A for receiving an instruction to start/stop measurement of blood pressure and a memory switch 52B for calling a past measurement result. These switches 52A and 52B input an operation signal corresponding to an instruction from a user to the CPU 110.

The memory 51 stores data of a program for controlling the sphygmomanometer 1, data used for controlling the sphygmomanometer 1, setting data for setting various functions of the sphygmomanometer 1, data of a measurement result of a blood pressure value, and the like. In addition, the memory 51 is used as a work memory or the like when a program is executed.

The power supply unit 53 supplies power to each unit of the sphygmomanometer 1 including the CPU 110, the first pressure sensor 30, the second pressure sensor 31, the pump 32, the discharge valve 33, the display 50, the memory 51, the A/D conversion circuits 300 and 310, the pump drive circuit 320, and the valve drive circuit 330.

The pump 32 supplies air as a fluid to the pressing cuff 23 through the second air pipe 38 in order to increase the pressure (this is represented by a reference sign “Pc”) of the pressing cuff 23 contained in the cuff 20. The discharge valve 33 is opened and closed to control the pressure Pc of the pressing cuff 23 by discharging or enclosing the air of the pressing cuff 23 through the second air pipe 38. The pump drive circuit 320 drives the pump 32 based on a control signal given from the CPU 110. The valve drive circuit 330 opens and closes the discharge valve 33 based on a control signal given from the CPU 110.

The first pressure sensor 30 is a piezoresistive pressure sensor in this example, and detects the pressure (this is represented by a reference sign “Ps”) of the sensing cuff 21 via the first air pipe 37. The pressure Ps of the sensing cuff 21 output from the first pressure sensor 30 is converted from an analog signal to a digital signal by the A/D conversion circuit 300 and input to the CPU 110. The second pressure sensor 31 is a piezoresistive pressure sensor like the first pressure sensor 30, and detects the pressure Pc of the pressing cuff 23 via the second air pipe 38. The pressure Pc of the pressing cuff 23 output from the second pressure sensor 31 is converted from an analog signal to a digital signal by the A/D conversion circuit 310 and input to the CPU 110.

The CPU 110 controls the operation of the entire sphygmomanometer 1 as a control unit. Specifically, the CPU 110 acts as a pressure control unit according to a program for controlling the sphygmomanometer 1 stored in the memory 51, and performs control to drive the pump 32 and the discharge valve 33 according to an operation signal from the operation unit 52. In addition, the CPU 110 acts as a blood pressure calculation unit to calculate a blood pressure value based on the data representing the pressure Ps of the sensing cuff 21 from the first pressure sensor 30 and the data representing the pressure Pc of the pressing cuff 23 from the second pressure sensor 31, and controls the display 50 and the memory 51. A specific procedures of blood pressure measurement will be described later.

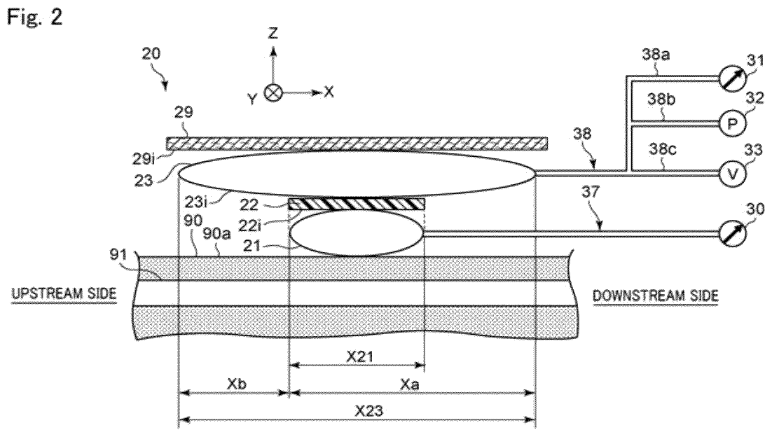

FIG. 2 illustrates a section of the cuff 20 (including the sensing cuff 21 and the pressing cuff 23) constituting the sphygmomanometer 1 in a state where the cuff 20 is attached to the measurement target site 90, together with the air pipe systems 37 and 38. As can be seen from FIG. 2, the cuff 20 mainly includes an outer cloth 29 having a belt-like shape positioned on the outermost circumference, the above-described pressing cuff 23 provided along a surface 29i on a side (inner circumferential side) facing the measurement target site 90 of the outer cloth 29, a back plate 22 provided along a surface 23i on a side (inner circumferential side) facing the measurement target site 90 of the pressing cuff 23, and the above-described sensing cuff 21 provided along a surface 22i on a side (inner circumferential side) facing the measurement target site 90 of the back plate 22.

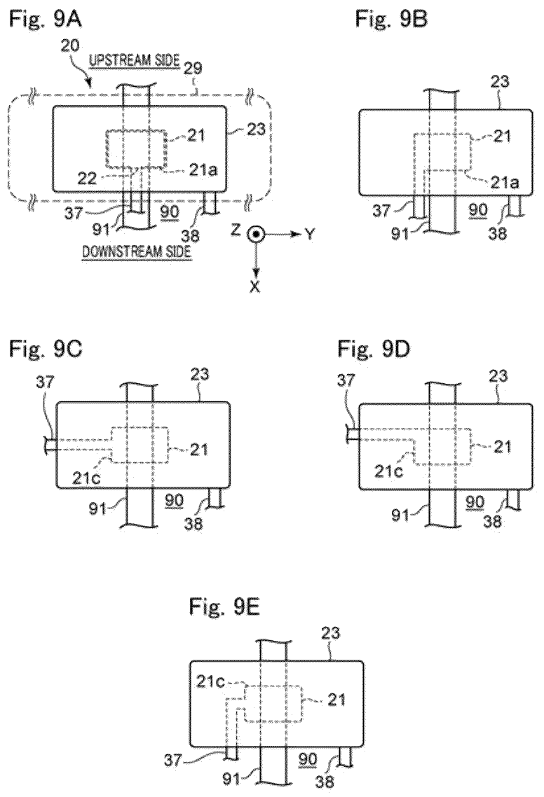

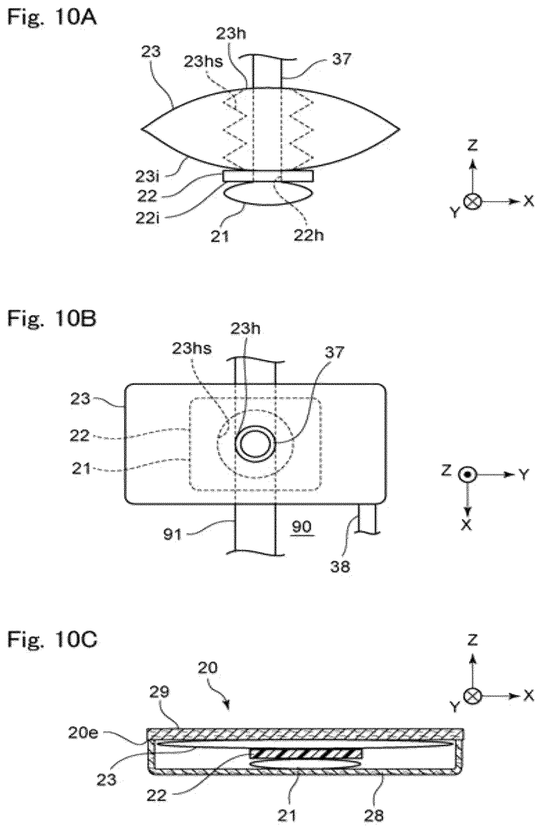

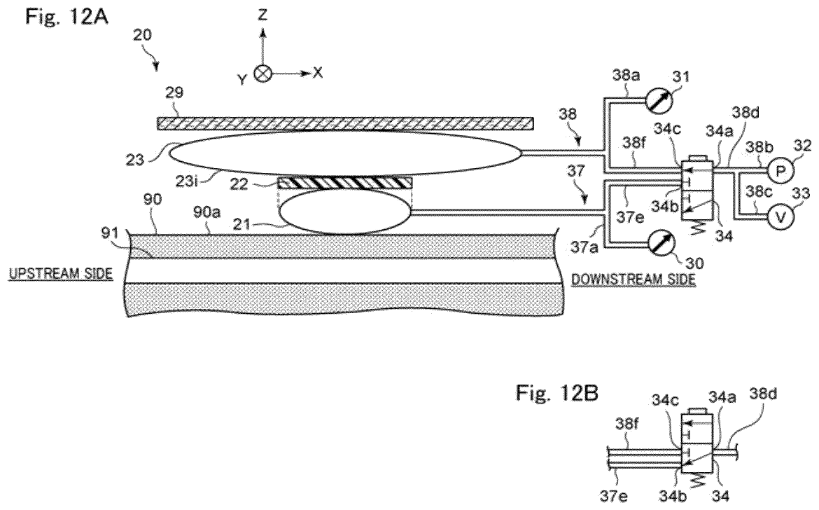

Here, for easy understanding, FIG. 2 also shows an XYZ orthogonal coordinate system indicating a “longitudinal direction Y”, a “width direction X”, and a “thickness direction Z” of the cuff 20. As for the cuff 20, the “longitudinal direction Y” means a direction in which the outer cloth 29 extends in a belt-like shape, and corresponds to the circumferential direction surrounding the measurement target site 90 in the attached state. The “width direction X” means a direction perpendicular to the longitudinal direction Y in a plane along the outer cloth 29, and corresponds to a direction in which an artery 91 passes through the measurement target site 90 in the attached state. “Upstream side” and “downstream side” mean upstream side and downstream side, respectively, with respect to the blood flow flowing through the artery 91. The “thickness direction Z” is a direction perpendicular to both the longitudinal direction Y and the width direction X (that is, outer cloth 29), and corresponds to a direction perpendicular to an outer circumferential surface 90a of the measurement target site 90 in the attached state. This XYZ orthogonal coordinate system is also shown in FIG. 9A, FIG. 10A, FIG. 10B, FIG. 10C, and FIG. 12A described later.

FIG. 9A schematically shows a planar layout of the cuff 20 in a state where the cuff 20 is unfolded. As shown in this planar layout, the outer cloth 29 has a belt-like shape (in this example, a rounded rectangle) extending in the longitudinal direction Y (the lateral direction in FIG. 9A) in this example. The outer cloth 29 can be curved or bent, but is configured not to substantially extend or contract in order to restrict the sensing cuff 21 and the pressing cuff 23 from expanding as a whole in a direction away from the measurement target site 90 during blood pressure measurement. Here, the “cloth” is not limited to a knitted cloth, and may include one layer or a plurality of layers of resin. In this example, the dimension of the outer cloth 29 in the longitudinal direction is set to be longer than a circumference length of the upper arm as the measurement target site 90.

The pressing cuff 23 has a rounded rectangular shape in a plane along the outer cloth 29. The planar dimension of the pressing cuff 23 is required to have a certain length and width in order to press the artery 91 and temporarily stop blood flow. In this example (an example of a cuff for an upper arm), the planar dimensions of the pressing cuff 23 are set to, for example, 24 cm in the longitudinal direction Y and 13 cm in the width direction X (indicated by X23 in FIG. 2). In this example, the pressing cuff 23 is formed in a bag shape by making a pair of sheets (in this example, stretchable polyurethane sheets) face to each other in the thickness direction Z and welding peripheral edges of the pair of sheets to each other with a near end of the second air pipe 38 interposed therebetween.

The back plate 22 is made of a plate-like resin (in this example, polypropylene) having a thickness of about 1 mm in this example. In this example, the shape and planar dimensions of the back plate 22 are set to be substantially the same as the shape and planar dimensions of the sensing cuff 21 described below, respectively. Since the back plate 22 is interposed between the pressing cuff 23 and the sensing cuff 21, the pressure Pc of the pressing cuff 23 can be reliably transmitted to the sensing cuff 21. Thus, the sensing cuff 21 can reliably compress the artery 91 of the measurement target site 90 via the air (pressure transmission fluid) contained in the sensing cuff 21 by the pressure Pc of the pressing cuff 23. At the same time, the back plate 22 blocks transmission of the pressure fluctuation component between the pressing cuff 23 and the sensing cuff 21. Thus, for example, it is possible to prevent mixing of a fluctuation component (caused by a pressure pulse wave from the artery 91, for example) included in the pressure Ps of the sensing cuff 21 as noise into the pressure Pc of the pressing cuff 23. Conversely, it is possible to prevent mixing of the fluctuation component included in the pressure Pc of the pressing cuff 23 as noise into the pressure Ps of the sensing cuff 21.

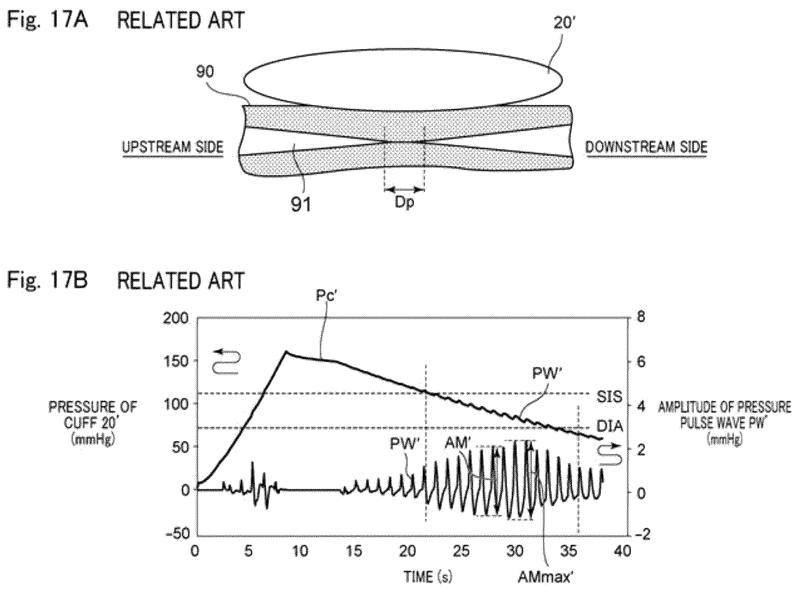

As illustrated in FIG. 9A, the sensing cuff 21 has a rectangular shape with rounded corners in a plane along the outer cloth 29 (and the pressing cuff 23) in this example. In this example, the sensing cuff 21 is formed in a bag shape by making a pair of sheets (in this example, a stretchable polyurethane sheet) face to each other in the thickness direction Z and welding peripheral edges of the pair of sheets to each other with a near end of the first air pipe 37 interposed therebetween. Here, the dimension of the sensing cuff 21 in the width direction X is desirably shorter than a pressure closing distance Dp (shown in FIG. 17A for a general cuff 20′) by which the artery 91 is compressed and closed when the pressure Pc of the pressing cuff 23 reaches a systolic blood pressure value SYS. In the example of FIG. 9A (an example of a cuff for an upper arm), the planar dimensions of the sensing cuff 21 are set to, for example, 7 cm in the longitudinal direction Y and 4 cm in the width direction X (indicated by X21 in FIG. 2). With this structure, as (an amplitude of) the pressure pulse wave acquired by the sensing cuff 21, only a volume change of the artery 91 in the place where the pressure Pc of the pressing cuff 23 is sufficiently applied is reflected. Thus, measurement accuracy of the blood pressure is improved.

In principle, the blood pressure can be measured when the planar dimension of the sensing cuff 21 is equal to or larger than the diameter of the artery 91. However, in consideration of occurrence of positional displacement when the cuff 20 is attached to the measurement target site 90, the planar dimension of the sensing cuff 21 is set to, for example, 1 cm×1 cm or more.

As shown in FIG. 2, in the width direction X along the direction in which the artery 91 of the measurement target site 90 passes, a range X21 occupied by the sensing cuff 21 is within a remaining ⅔ range Xa of the width direction dimension X23 of the pressing cuff 23 excluding a predetermined ⅓ range Xb from the upstream side of the artery 91. Assume that, with regard to the width direction X of the pressing cuff 23, the range X21 occupied by the sensing cuff 21 is within a range Xb of ⅓ of the width direction dimension X23 of the pressing cuff 23 from the upstream side of the artery 91. Then, for example, even when the pressure Pc of the pressing cuff 23 is in the pressure section higher than the systolic blood pressure value SYS and the blood flow of the artery 91 is stopped in the depressurization process, there is a possibility that a pulse wave from the upstream side of the artery 91 to immediately below the pressing cuff 23 is mixed as noise into the pressure Ps of the sensing cuff 21 detected by the first pressure sensor 30. Thus, in this example, with regard to the width direction X of the pressing cuff 23, the range X21 occupied by the sensing cuff 21 is set within the remaining ⅔ range Xa excluding the range Xb of ⅓ from the upstream side of the artery 91, of the width direction dimension of the pressing cuff 23. Thus, for example, when the pressure Pc of the pressing cuff 23 is in the pressure section higher than the systolic blood pressure value SYS and the blood flow of the artery 91 is stopped in the depressurization process, it is possible to prevent mixing of the pulse wave from the upstream side of the artery 91 toward immediately below the pressing cuff 23 as noise into the pressure Ps of the sensing cuff 21 detected by the first pressure sensor 30.

In the examples of FIGS. 2 and 9A, the first air pipe 37 and the second air pipe 38 are drawn to the outside along the width direction X of the cuff 20 from the sides corresponding to the downstream sides of the sensing cuff 21 and the pressing cuff 23, respectively, so as not to interfere with the measurement as much as possible.

As shown in FIG. 10C, the outer cloth 29 and a stretchable inner cloth 28 may be formed in a bag shape so as to face to each other, and the pressing cuff 23, the back plate 22, and the sensing cuff 21 may be integrally contained in the bag. The peripheral edge portion of the outer cloth 29 and a peripheral edge portion of the inner cloth 28 (that is, a peripheral edge portion 20e of the cuff 20) are attached to each other by sewing or welding with the first air pipe 37 and the second air pipe 38 interposed therebetween. This enables the cuff 20 to be handled integrally, and the sensing cuff 21 and the like contained in the bag can be protected.

(Blood Pressure Measurement Method)

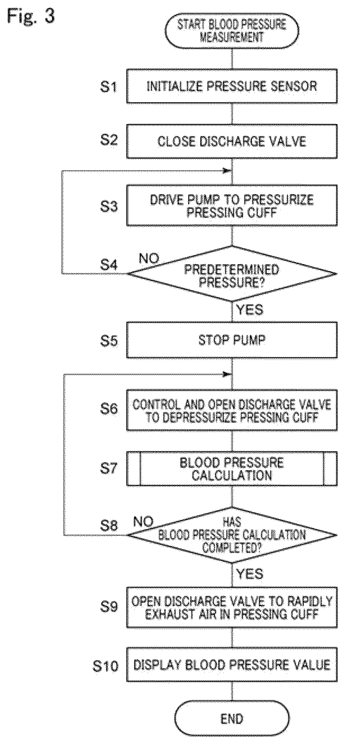

FIG. 3 shows a schematic operation flow of blood pressure measurement by the sphygmomanometer 1. This operation flow is a flow for calculating the blood pressure in the depressurization process.

When a user (subject) turns on the measurement switch 52A provided on the main body 10 to instruct to start the measurement in the attached state (see FIG. 2) in which the cuff 20 is attached to the measurement target site (in this example, an upper arm) 90, the CPU 110 initializes the pressure sensor as shown in step S1 of FIG. 3. Specifically, the CPU 110 initializes the processing memory area, stops the pump 32, and performs 0 mmHg adjustment (the atmospheric pressure is set to 0 mmHg) of the first pressure sensor 30 and the second pressure sensor 31 in a state where the discharge valve 33 is opened.

In this example, it is assumed that air as a pressure transmission fluid is sealed in a predetermined amount in the sensing cuff 21 (and the first air pipe 37) at the manufacturing stage of the sphygmomanometer 1. Thus, for example, it is possible to omit time and effort for enclosing the air in the sensing cuff 21 every time the blood pressure is measured. Here, the “predetermined amount” refers to, for example, an amount that can avoid a situation in which the bag forming the sensing cuff 21 is crushed in the thickness direction Z by the pressure Pc of the pressing cuff 23 and the sheet forming the bag is brought into close contact. Avoiding such a situation enables the sensing cuff 21 to reliably compress the artery 91 of the measurement target site 90 via the air as the pressure transmission fluid with the pressure Pc of the pressing cuff 23, and to reliably receive a pressure pulse wave from the artery 91.

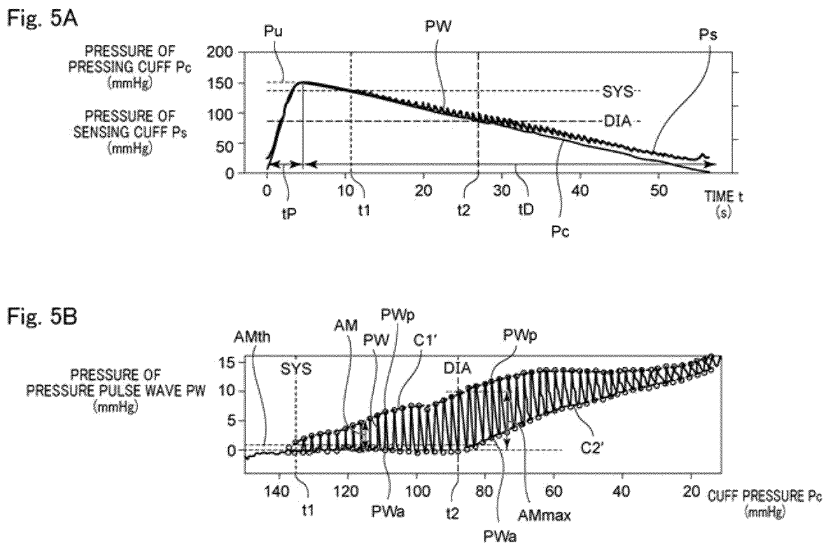

Next, in step S2 of FIG. 3, the CPU 110 closes the discharge valve 33 via the valve drive circuit 330. Subsequently, in step S3, the CPU 110 acts as a pressure control unit, drives the pump 32 via the pump drive circuit 320, and starts pressurization of the cuff 20 (to be precise, the pressing cuff 23) (pressurization process). The CPU 110 controls the pressurization speed to be substantially constant (for example, within a range of 20 mmHg/sec to 40 mmHg/sec) based on the output of the second pressure sensor 31 while supplying air from the pump 32 to the pressing cuff 23 through the second air pipe 38. This causes the pressure Pc of the pressing cuff 23 to increase substantially linearly as indicated by a pressurization period tP in FIG. 5A. Since the sensing cuff 21 is pressed by the pressing cuff 23 toward the measurement target site 90 via the back plate 22, the pressure Ps of the sensing cuff 21 also increases with the pressure Pc of the pressing cuff 23.

Next, in step S4 of FIG. 3, the CPU 110 determines whether the pressure Pc of the pressing cuff 23 has reached a predetermined pressure (indicated by Pu in FIG. 5A). Here, it is assumed that the predetermined pressure Pu is set sufficiently higher than an assumed systolic blood pressure value SYS of the subject. In this example, the predetermined pressure Pu is set to 150 mmHg. When the pressure Pc of the pressing cuff 23 has not reached the predetermined pressure Pu (NO in step S4 in FIG. 3), the CPU 110 returns to step S3 and continues the pressurization. When the pressure Pc of the pressing cuff 23 reaches the predetermined pressure Pu (YES in step S4 in FIG. 3), the CPU 110 stops the pump 32 in step S5.

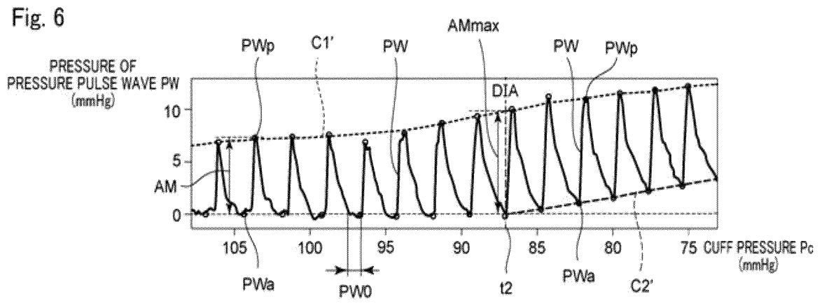

Next, in step S6 of FIG. 3, the CPU 110 acts as a pressure control unit, and gradually opens the discharge valve 33 via the valve drive circuit 330. This causes the CPU 110 to gradually reduce the pressure Pc of the pressing cuff 23 at a constant speed (in this example, 5 mmHg/sec) as indicated by a depressurization period tD in FIG. 5A (depressurization process) based on the output of the second pressure sensor 31. In the depressurization process, as shown in step S7 of FIG. 3, the CPU 110 acts as a blood pressure calculation unit, and attempts to calculate the blood pressure value (the systolic blood pressure value SYS and the diastolic blood pressure value DIA) based on the data representing the pressure Ps of the sensing cuff 21 from the first pressure sensor 30 and the data representing the pressure Pc of the pressing cuff 23 from the second pressure sensor 31 acquired by this time. That is, the CPU 110 subtracts the direct current component of the data representing the pressure Pc of the pressing cuff 23 (which may be extracted from raw data via a low-pass filter) from the data representing the pressure Ps of the sensing cuff 21 in the depressurization process while associating the times of generation of those data with each other, and obtains the pressure at a rising start point PWa and the pressure at a peak point PWp indicated by the pressure pulse wave PW for each beat as shown in FIGS. 5B and 6. Then, as shown in FIGS. 5B and 6, in the depressurization process, the CPU 110 obtains the pressure Pc of the pressing cuff 23 at a first time point t1 at which the pressure at the peak point PWp for each beat starts to show a positive value from the zero level as the systolic blood pressure value SYS, and obtains the pressure Pc of the pressing cuff 23 at a second time point t2 at which the pressure at the rising start point PWa for each beat starts to show a positive value from the zero level as the diastolic blood pressure value DIA (FIG. 6 is an enlarged view of the vicinity of the portion corresponding to the second time point t2 in FIG. 5B, in other words, the vicinity of the portion where the cuff pressure Pc on the horizontal axis corresponds to the diastolic blood pressure value DIA). The principle of blood pressure calculation and a more specific way of blood pressure calculation will be described in detail later.

At this time point, in a case where the blood pressure value cannot be calculated yet due to a shortage of data (NO in step S8 in FIG. 3), the processing of steps S6 to S8 is repeated.

When the blood pressure value can be calculated in this way (YES in step S8), the CPU 110 performs control to open the discharge valve 33 via the valve drive circuit 330 and rapidly exhaust the air in the pressing cuff 23 in step S9. Further, in step S10, the CPU 110 performs control to display the measurement result of the blood pressure value on the display 50 and store the measurement result of the blood pressure value in the memory 51.

(Principle of Blood Pressure Calculation)

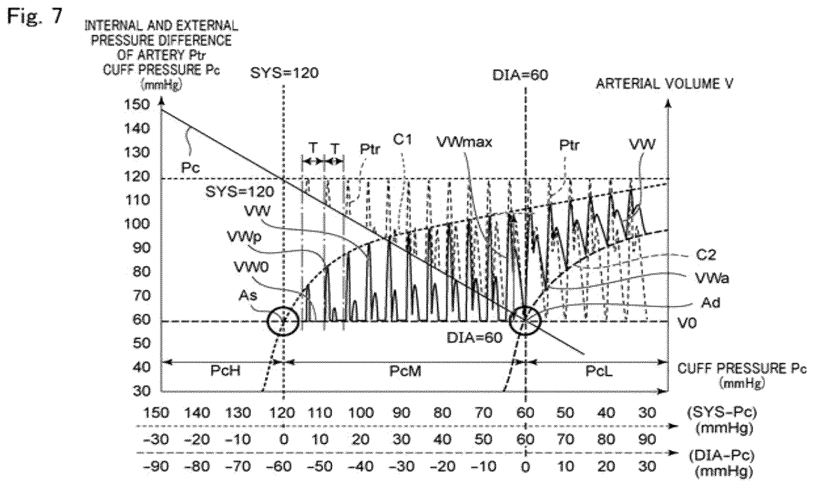

In principle, how to calculate the blood pressure by the sphygmomanometer 1 is described as follows. For example, FIG. 7 schematically shows how to calculate the blood pressure in the depressurization process when the systolic blood pressure value SYS and the diastolic blood pressure value DIA of the measurement target site 90 of the subject are assumed to be 120 mmHg and 60 mmHg, respectively. In FIG. 7, the left vertical axis represents the direct current component (for the sake of simplicity, this direct current component is referred to as a “cuff pressure Pc” using the same reference numeral) of the pressure Pc of the pressing cuff 23 and an internal and external pressure difference Ptr of the artery of the measurement target site 90. The right vertical axis represents an arterial volume V. The horizontal axis represents the cuff pressure Pc. In this example, it is assumed that the rate of depressurization by the pressure control unit is constant (in this example, 5 mmHg/sec). Thus, the horizontal axis also corresponds to the time t.

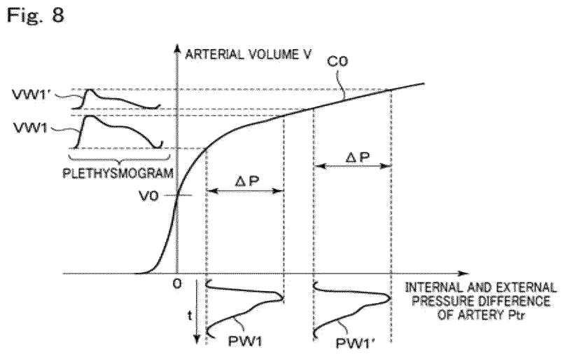

Here, the internal and external pressure difference Ptr of the artery is defined as a difference between the internal pressure (represented as “Pa”) of the artery of the measurement target site 90 and the cuff pressure Pc. That is, Ptr=Pa−Pc. Here, it is generally known that there is a correspondence relationship (called “tube law”) shown by a curve C0 in FIG. 8 between the internal and external pressure difference Ptr of the artery and a volume (referred to as “arterial volume V”) of the artery (blood vessel). As the internal and external pressure difference Ptr of the artery increases positively, the arterial volume V increases monotonically and gradually saturates. Even when the pulse waves PW1 and PW1′ having the same wave height ΔP are applied to the artery, the generated plethysmograms VW1 and VW1′ vary according to the tube law (curve C0), since the internal and external pressure difference Ptr of the artery varies as the cuff pressure Pc varies. Here, when the internal and external pressure difference of the artery Ptr=0, that is, when the internal pressure Pa of the artery and the cuff pressure Pc are balanced, the arterial volume is V0 (this is called “equilibrium level”).

In the depressurization process shown in FIG. 7, in the pressure section PcH in which the cuff pressure Pc is higher than the systolic blood pressure value SYS (in this example, 120 mmHg), the internal and external pressure difference of the artery Ptr<0 is always satisfied regardless of the pulse wave (beat). This completely stops blood flow through the artery 91. Thus, the arterial volume V does not change and does not rise from the equilibrium level V0.

It is assumed that the pressure control unit advances the depressurization and the cuff pressure Pc enters the pressure section PcM between the systolic blood pressure value SYS (in this example, 120 mmHg) and the diastolic blood pressure value DIA (in this example, 60 mmHg). The blood flows from the upstream side to the downstream side from a point (referred to as a “blood flow restart point As”) where the cuff pressure Pc falls below the systolic blood pressure value SYS. In the pressure section PcM, the internal and external pressure difference of the artery Ptr>0 is satisfied in a period (partial period in the cycle T of one beat) in which the internal pressure Pa of the artery exceeds the cuff pressure Pc in the cycle T of one beat. In FIG. 7, the positive internal and external pressure difference Ptr of the artery is represented by a broken line. When the internal and external pressure difference of the artery Ptr>0 is satisfied, a plethysmogram VW appears with a peak as indicated by a solid line in FIG. 7 according to the tube law (the curve C0 in FIG. 8). The plethysmogram VW is detected as a fluctuation component of the pressure Ps of the sensing cuff 21 (the pressure pulse wave PW shown in FIGS. 5B and 6). In the remaining period of the cycle T of one beat, the internal and external pressure difference of the artery Ptr<0 is satisfied. Thus, the plethysmogram VW includes a flat portion VW0 (a portion indicating the equilibrium level V0). Accordingly, as shown in FIG. 6, the pressure of the pressure pulse wave PW detected by the sensing cuff 21 also includes the flat portion PW0.

Here, a curve C1 shown in FIG. 7 connecting the upper peak (peak point) VWp of the plethysmogram VW represents a relationship between the pressure amount (SYS Pc) obtained by subtracting the cuff pressure Pc from the systolic blood pressure value SYS (=120 mmHg) and the arterial volume V according to the above tube law (the curve C0 in FIG. 8). That is, in the pressure section PcM (and the pressure section PeL described below), as the cuff pressure Pc decreases, the upper peak VWp of the plethysmogram VW increases along the curve C1 for each beat. In response to this, the upper peak (peak point) PWp indicated by the pressure of the pressure pulse wave PW shown in FIGS. 5B and 6 also rises along a first curve C1′ (corresponding to the curve C1 in FIG. 7) for each beat (in principle, it increases monotonically and gradually saturates). Here, the first curve C1′ is defined as a curve connecting the peak points PWp (or a curve approximating the curve).

As can be understood from the above description, in the depressurization process, the first time point (that is, the time point at which the first curve C1′ transitions from the zero level to the positive value) t1 at which the pressure at the peak point PWp for each beat of the pressure pulse wave PW starts to show a positive value from the zero level shown in FIG. 5B corresponds to the time point at which the blood flow restarts, that is, the point at which the cuff pressure Pc falls below the systolic blood pressure value SYS (the blood flow restart point As in FIG. 7). Thus, the cuff pressure Pc at the first time point t1 corresponds to the systolic blood pressure value SYS in principle.

In FIG. 7, it is assumed that the depressurization by the pressure control unit further proceeds and the cuff pressure Pc enters the pressure section PeL lower than the diastolic blood pressure value DIA (in this example, 60 mmHg). The artery is completely opened from the point (referred to as a “DIA restart point Ad”) where the cuff pressure Pc falls below the diastolic blood pressure value DIA. In this range PcL, the flat portion VW0 included in the plethysmogram VW in the previous range PcM disappears, and the waveform of the plethysmogram VW becomes a sharpened complete waveform. Here, a curve C2 connecting the lower peak (rising start point) VWa of the plethysmogram VW shown in FIG. 7 represents the relationship between the pressure amount (DIA−Pc) obtained by subtracting the cuff pressure Pc from the diastolic blood pressure value DIA (=60 mmHg) and the arterial volume V according to the tube law (the curve C0 in FIG. 8). That is, in this range PcL, as the cuff pressure Pc decreases, the lower peak (rising start point) VWa of the plethysmogram VW rises along the curve C2 for each beat. In response to this, the lower peak (rising start point) PWa indicated by the pressure of the pressure pulse wave PW shown in FIGS. 5B and 6 also rises along the second curve C2′ (corresponding to the curve C2 in FIG. 7) for each beat (in principle, it monotonically increases and gradually saturates). Here, the second curve C2′ is defined as a curve connecting the rising start point PWa (or a curve approximating the curve).

As can be understood from the above description and as shown in FIGS. 5B and 6, the second time point (that is, the time point at which the second curve C2′ transitions from the zero level to the positive value) t2 at which the pressure at the rising start point PWa for each beat of the pressure pulse wave PW starts to show a positive value from the zero level corresponds to a point at which the cuff pressure Pc falls below the diastolic blood pressure value DIA (the DIA restart point Ad in FIG. 7). Thus, the cuff pressure Pc at the second time point t2 corresponds to the diastolic blood pressure value DIA in principle.

Here, in FIG. 7, the curve C2 corresponds to the curve C1 shifted to the right by a difference (SYS−DIA) between the systolic blood pressure value SYS and the diastolic blood pressure value DIA. According to the tube law shown in FIG. 8, the curves C1 and C2 rise from the blood flow restart point As and the DIA restart point Ad, respectively, and both monotonically increase and gradually saturate. Thus, at the DIA restart point Ad, the amplitude of the plethysmogram VW indicates a maximum value VWmax. Accordingly, an amplitude (called “pulse wave amplitude AM”) of the pressure pulse wave PW shown in FIGS. 5B and 6 also indicates a maximum value AMmax. Thus, in principle, the cuff pressure Pc at the time point (the second time point t2) at which the pulse wave amplitude AM indicates the maximum value AMmax can be correctly obtained as the diastolic blood pressure value DIA. As shown in FIG. 6, the pulse wave amplitude AM is defined as a difference between the pressure at the peak point PWp of the pressure pulse wave PW and the pressure at the rising start point PWa.

As shown in FIG. 17A, a general cuff 20′ does not include the sensing cuff 21 in the present embodiment but includes a single fluid bag corresponding to the pressing cuff 23. In a general oscillometric method, a fluctuation component (pulse wave amplitude AM′) of the pressure pulse wave PW′ is obtained from the pressure Pc ‘of the cuff 20’ through a high-pass filter as shown in FIG. 17B. An envelope is set for a row of the pulse wave amplitudes AM′, threshold levels (including a threshold level for systole and a threshold level for diastole) of a predetermined proportion (ratio) with respect to a maximum value (corresponding to AMmax′) of the envelope is set, and cuff pressures at the times when the envelope crosses these threshold levels are calculated as the systolic blood pressure value SYS and the diastolic blood pressure value DIA, respectively. In the way using the general cuff 20′, the second time point t2 (that is, the time point at which the second curve C2′ shown in FIGS. 5B and 6 transitions from the zero level to the positive value) shown in FIGS. 5B and 6 does not appear. Thus, in the method using the general cuff 20′, the diastolic blood pressure value DIA cannot be obtained by the above-described principle.

(Specific Way of Calculating Blood Pressure)

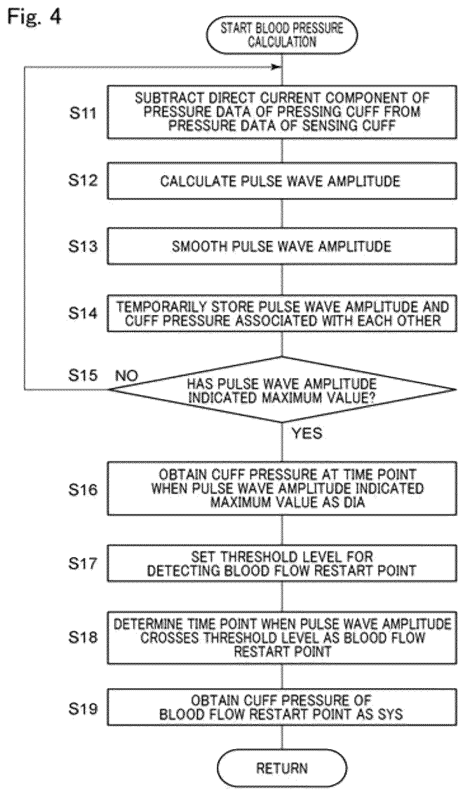

FIG. 4 shows a detailed flow of how to calculate the blood pressure in step S7 of FIG. 3 according to the principle of the blood pressure calculation of the present invention described above.

In step S11 of FIG. 4, the CPU 110 subtracts the direct current component (cuff pressure Pc) of the pressure Pc of the pressing cuff 23 through the second pressure sensor 31 from the data representing the pressure Ps of the sensing cuff 21 through the first pressure sensor 30 while associating the times of generation of the data with each other, and obtains the pressure at the rising start point PWa and the pressure at the peak point PWp indicated by the pressure pulse wave PW for each beat as shown in FIG. 5B and FIG. 6.

Next, in step S12 of FIG. 4, the CPU 110 calculates a difference obtained by subtracting the pressure at the rising start point PWa from the pressure at the peak point PWp for each beat as the pulse wave amplitude AM shown in FIGS. 5B and 6.

Next, in step S13 of FIG. 4, in order to accurately obtain the systolic blood pressure value SYS and the diastolic blood pressure value DIA, the CPU 110 takes a moving average over a plurality of predetermined beats (five beats in this example) and smooths the pulse wave amplitude AM for each beat. Then, in step S14, the CPU 110 stores the smoothed data of each pulse wave amplitude AM in the memory 51 in time series in association with the data representing the cuff pressure Pc at the time point when the pulse wave amplitude AM is indicated. In this example, the cuff pressure Pc is extracted as a direct current component from raw data representing the pressure Pc of the pressing cuff 23 via a low-pass filter.

Next, in step S15 of FIG. 4, the CPU 110 refers to the contents stored in the memory 51 and determines whether the pulse wave amplitude AM has indicated the maximum value AMmax shown in FIGS. 5B and 6. At this time point, when the pulse wave amplitude AM has not yet indicated the maximum value AMmax (NO in step S15 in FIG. 4), the processing of steps S11 to S15 is repeated. On the other hand, when the pulse wave amplitude AM has indicated the maximum value AMmax (YES in step S15 in FIG. 4), the process proceeds to step S16, and the CPU 110 determines the time point at which the pulse wave amplitude AM indicated the maximum value AMmax as the second time point t2 (that is, the time point at which the second curve C2′ shown in FIGS. 5B and 6 transitioned from the zero level to the positive value), and obtains the cuff pressure Pc corresponding to the determined second time point t2 as the diastolic blood pressure value DIA. Here, whether the pulse wave amplitude AM has indicated the maximum value AMmax is determined by comparison between the pulse wave amplitudes AM, and thus can be determined with higher accuracy as compared with the case of comparison with a zero level (buried in noise). Thus, in the sphygmomanometer 1, the second time point t2 can be accurately determined, whereby the diastolic blood pressure value DIA can be accurately obtained.

Next, in step S17 of FIG. 4, the CPU 110 multiplies the maximum value AMmax of the pulse wave amplitude AM by a predetermined ratio, and sets a threshold level AMth for determining the first time point t1 (that is, the time point at which the first curve C1′ shown in FIG. 5B transitioned from the zero level to a positive value) as shown in FIG. 5B. Here, the “predetermined ratio” is set to exceed the actual noise level and be as small as possible in order to accurately determine the first time point t1. The “predetermined ratio” is set to, for example, about 0.1 with respect to the maximum value AMmax of the pulse wave amplitude AM.

Subsequently, in step S18 in FIG. 4, the CPU 110 refers to the contents stored in the memory 51, and determines a time point at which the pulse wave amplitude AM crosses the threshold level AMth (indicating the blood flow restart point) in the depressurization process as the first time point t1. More specifically, among the data in which the pulse wave amplitudes AM exceeded the threshold level AMth before the second time point t2, the data in which the corresponding cuff pressure Pc was the maximum is searched, and the time point indicated by the data is determined as the first time point t1. Then, in step S19 of FIG. 4, the CPU 110 obtains the cuff pressure Pc corresponding to the determined first time point t1 as the systolic blood pressure value SYS. Here, whether the pulse wave amplitude AM exceeds the threshold level AMth is determined by comparison between the pulse wave amplitudes AM and the set threshold level AMth, and thus can be determined with higher accuracy as compared with the case of comparison with a zero level (buried in noise). Thus, in the sphygmomanometer 1, the first time point t1 can be accurately determined, whereby the systolic blood pressure value SYS can be accurately obtained.

In this way, according to the sphygmomanometer 1, the blood pressure at the measurement target site 90 can be correctly measured in principle in a noninvasive manner.

(First Modification)

In the example of FIG. 9A, the first air pipe 37 is drawn out from substantially the center of the side 21a on the downstream side (+X side) of the sensing cuff 21 to the outside along the width direction X of the cuff 20. However, the present invention is not limited to this configuration. As arrangements of the first air pipe 37, as shown in FIGS. 9B to 9E, various manners of being deviated from the artery 91 in a plan view viewed from the thickness direction Z perpendicular to (the outer circumferential surface 90a of) the measurement target site 90 are considered (for the sake of simplicity, the outer cloth 29 and the back plate 22 are not shown in FIGS. 9B to 9E).

Specifically, in the example of FIG. 9B, the first air pipe 37 is drawn out in parallel away from the artery 91 from the end of the side 21a on the downstream side of the sensing cuff 21. In the examples of FIGS. 9C and 9D, the first air pipe 37 is drawn to the outside along the longitudinal direction Y of the cuff 20 from substantially the center and the end of the side on the −Y side of the sensing cuff 21, respectively. In the example of FIG. 9E, the first air pipe 37 slightly extends in the longitudinal direction Y from substantially the center of the side on the −Y side, is bent in the width direction X, and is drawn out in parallel away from the artery 91. In these examples, the first air pipe 37 is drawn out of the artery 91 to the outside of the cuff 20 (in other words, the region where the pressing cuff 23 surrounds the measurement target site 90). Thus, the first air pipe 37 does not prevent the pressure of the pressing cuff 23 from compressing the artery 91 of the measurement target site 90 via the sensing cuff 21. In addition, the first air pipe 37 does not prevent the sensing cuff 21 from receiving the pressure pulse wave PW from the artery 91.

(Second Modification)

In the examples of FIGS. 9A to 9E, the first air pipe 37 is drawn to the outside of the cuff 20 in a plane (XY plane) along the outer cloth 29 (and the pressing cuff 23), but the present invention is not limited to this configuration. As shown in the sectional view of FIG. 10A and the plan view of FIG. 10B, the first air pipe 37 may penetrate the pressing cuff 23 and be drawn out of the cuff 20 in the thickness direction Z (for simplicity, outer cloth 29 is not shown in FIGS. 10A and 10B).

Specifically, in the second modification, as shown in FIG. 10A, the pressing cuff 23 is provided with a through hole 23h penetrating in the thickness direction Z. As shown in FIG. 10B, the through hole 23h is provided at a central portion in the XY plane of the pressing cuff 23 (corresponding to a portion in a region facing the sensing cuff 21). Accordingly, as shown in FIG. 10A, a cylindrical through hole 22h penetrating in the thickness direction Z is also provided at a central portion in the XY plane of the back plate 22. The inner diameters of the through holes 22h and 23h are set to be slightly larger than the outer diameter of the first air pipe 37 so that the first air pipe 37 can pass therethrough. The first air pipe 37 extends upward (+Z direction) from the upper surface of the sensing cuff 21 in FIG. 10A, and is drawn out of the cuff 20 through the through holes 22h and 23h. In the second modification as well, as in the first modification, the first air pipe 37 does not prevent the pressure of the pressing cuff 23 from compressing the artery 91 of the measurement target site 90 via the sensing cuff 21. In addition, the first air pipe 37 does not prevent the sensing cuff 21 from receiving the pressure pulse wave PW from the artery 91.

In the second modification, an inner peripheral surface 23hs of the through hole 23h of the pressing cuff 23 is formed in a bellows shape. Thus, it is possible to facilitate expansion and contraction of the pressing cuff 23 in the thickness direction Z.

(Third Modification)

In the above example, air as a pressure transmission fluid is sealed in a predetermined amount in the sensing cuff 21 (and the first air pipe 37) at the manufacturing stage of the sphygmomanometer 1. However, the present invention is not limited to this configuration. Every time the blood pressure is measured, air as a pressure transmission fluid may be enclosed in the sensing cuff 21, and the air may be discharged from the sensing cuff 21 after a completion of calculation of the blood pressure value.

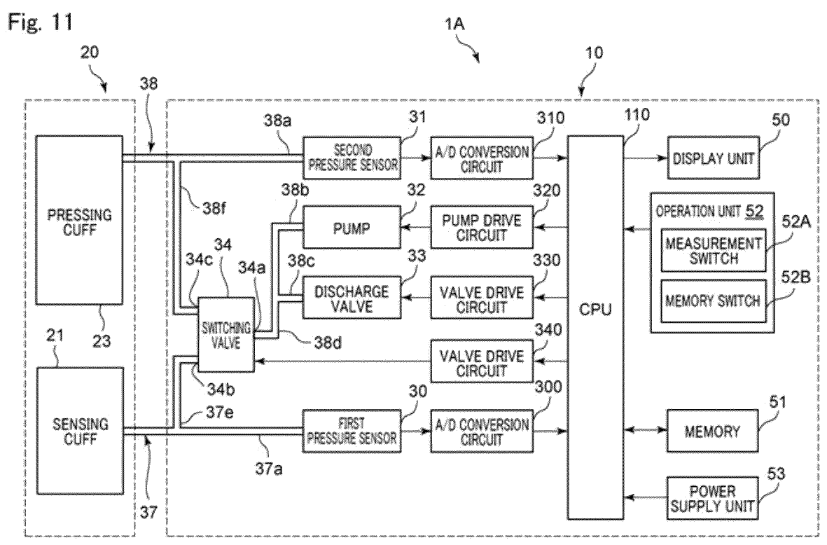

FIG. 11 shows a block configuration of a modification (this is referred to as a “sphygmomanometer 1A”) in which the sphygmomanometer 1 is modified in such a manner. In the sphygmomanometer 1A, in addition to the components of the sphygmomanometer 1, a switching valve 34 that is a three-port two-position electromagnetic valve and a valve drive circuit 340 that drives the switching valve 34 are mounted on the main body 10. In FIG. 11, the same components as those in FIG. 1 are denoted by the same reference numerals, and redundant description is omitted. In particular, the cuff 20 is the same as the cuff 20 shown in FIG. 1.

To one port 34a of the switching valve 34, an air pipe 38b connected to the pump 32 and an air pipe 38c connected to the discharge valve 33 are joined into one pipe and connected in a fluid communicable manner as an air pipe 38d. Air pipes 37e and 38f are connected to the remaining ports 34b and 34c of the switching valve 34, respectively, in a fluid communicable manner. These air pipes 37e and 38f are connected to the first air pipe 37 and the second air pipe 38, respectively, in a fluid communicable manner. In this example, the valve drive circuit 340 switches the switching valve 34 between a rest position (at the time of non-energization) as a first position shown in FIG. 12A and an operation position (at the time of energization) as a second position shown in FIG. 12B based on a control signal given from the CPU 110. Here, FIG. 12A shows a section in a state where the cuff 20 constituting the sphygmomanometer 1A is attached to the measurement target site 90 together with the air pipe systems 37 and 38, corresponding to FIG. 2.

The switching valve 34 connects the air pipe 38d and the air pipe 38f in a fluid communicable manner at the rest position shown in FIG. 12A. This causes the pump 32 and the discharge valve 33, and the pressing cuff 23 and the second pressure sensor 31 to be connected via the air pipes 38b, 38c, 38d, 38f, and 38 in a fluid communicable manner. At this time, the sensing cuff 21 is separated from the pump 32 and the discharge valve 33 and sealed together with the first pressure sensor 30. On the other hand, the switching valve 34 connects the air pipe 38d and the air pipe 37e in a fluid communicable manner at the operating position shown in FIG. 12B. This causes the pump 32 and the discharge valve 33, and the sensing cuff 21 and the first pressure sensor 30 to be connected via the air pipes 38b, 38c, 38d, 37e, and 37 in a fluid communicable manner. At this time, the pressing cuff 23 is separated from the pump 32 and the discharge valve 33 and sealed together with the second pressure sensor 31.

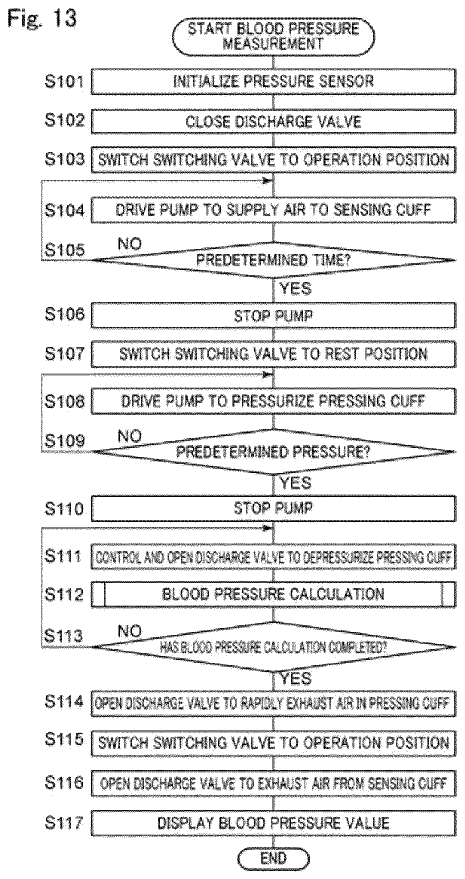

FIG. 13 shows a schematic flow of blood pressure measurement by the sphygmomanometer 1A.

When the user (subject) turns on the measurement switch 52A provided on the main body 10 to instruct to start the measurement in the attached state (see FIG. 12A) in which the cuff 20 is attached to the measurement target site (in this example, an upper arm) 90, the CPU 110 initializes the pressure sensor as shown in step S101 of FIG. 13. This initialization processing is the same as that in step S1 in FIG. 3.

Next, in step S102 in FIG. 13, the CPU 110 closes the discharge valve 33. Subsequently, in step S103, the CPU 110 switches the switching valve 34 to the operation position shown in FIG. 12B. Thereby, the pump 32 and the discharge valve 33, and the sensing cuff 21 and the first pressure sensor 30 are connected via the air pipes 38b, 38c, 38d, 37e, and 37 in a fluid communicable manner.

Next, in step S104 of FIG. 13, the CPU 110 acts as a fluid containment control unit, drives the pump 32, and supplies air as a pressure transmission fluid to the sensing cuff 21 through the air pipes 38h, 38d, 37e, and 37 in this order. Next, in step S105, the CPU 110 determines whether a predetermined time (for example, it is set within a range of 3 seconds to 10 seconds) has elapsed. Here, if the predetermined time has not yet elapsed (NO in step S105), the process returns to step S104, and the supply of air is continued until the predetermined time elapses. When the predetermined time has elapsed (YES in step S105), the process proceeds to step S106 to stop the pump 32. In this way, the air as the pressure transmission fluid can be automatically enclosed in the sensing cuff 21 by a predetermined amount in advance of a start of pressurization of the pressing cuff 23. Here, the “predetermined amount” refers to, as in the previous example, an amount that can avoid a situation in which the bag forming the sensing cuff 21 is crushed in the thickness direction Z by the pressure Pc of the pressing cuff 23, and the sheet forming the bag is brought into close contact. Avoiding such a situation enables the sensing cuff 21 to reliably compress the artery 91 of the measurement target site 90 via the air as the pressure transmission fluid with the pressure Pc of the pressing cuff 23, and to reliably receive a pressure pulse wave from the artery 91.

Next, in step S107 in FIG. 13, the CPU 110 acts as a fluid containment control unit to switch the switching valve 34 to the rest position shown in FIG. 12A. Thereby, the pump 32 and the discharge valve 33, and the pressing cuff 23 and the second pressure sensor 31 are connected via the air pipes 38b, 38c, 38d, 38f, and 38 in a fluid communicable manner. At this time, the sensing cuff 21 is separated from the pump 32 and the discharge valve 33, and sealed together with the first pressure sensor 30. Thus, the flow paths formed by the air pipe systems 37 and 38 are substantially in the same state as the flow paths shown in FIG. 1. This state is maintained from step S108 to step S114 in FIG. 13 described below.

Next, from step S108 to step S114 in FIG. 13, the CPU 110 serves as a pressure control unit and a blood pressure calculation unit, and advances the processing in the same way as in step S3 to step S9 in FIG. 3. As a result, the blood pressure value (the systolic blood pressure value SYS and the diastolic blood pressure value DIA) of the measurement target site 90 is calculated correctly in principle.

Next, in step S115 of FIG. 13, the CPU 110 acts as a fluid containment control unit to switch the switching valve 34 to the operating position shown in FIG. 1211. Thereby, the pump 32 and the discharge valve 33, and the sensing cuff 21 and the first pressure sensor 30 are connected via the air pipes 38b, 38c, 38d, 37e, and 37 in a fluid communicable manner.

Next, in step S116 in FIG. 13, the CPU 110 acts as a fluid containment control unit to open the discharge valve 33. This causes the air as the pressure transmission fluid is discharged from the sensing cuff 21 through the air pipes 37, 37e, 38d, and 38c and the discharge valve 33 in this order. Thereafter, in step S117, the CPU 110 performs control to display the measurement result of the blood pressure value on the display 50 and store the measurement result of the blood pressure value in the memory 51.

In this way, according to the sphygmomanometer 1A, it is possible to automatically enclose the predetermined amount of the air as the pressure transmission fluid in the sensing cuff 21 in advance of a start of pressurization of the pressing cuff 23 every time the blood pressure is measured with a relatively small number of components. In addition, the air as the pressure transmission fluid can be automatically discharged from the sensing cuff 21 after a completion of the calculation of the blood pressure value.

Further, in the sphygmomanometer 1A, the pump 32 and the discharge valve 33 are connected to the sensing cuff 21 and the first pressure sensor 30 in a fluid communicable manner (the switching valve 34 takes the operation position shown in FIG. 12B) only for a temporary period of steps S103 to S106 and steps S115 to S116. Thus, power consumption related to the switching valve 34 can be saved.

(Fourth Modification)

In the above example, the direct current component (the cuff pressure Pc) of the pressure Pc of the pressing cuff 23 serving as the reference of the blood pressure calculation is extracted from raw data via a low-pass filter, but the present invention is not limited to this configuration. For example, in the depressurization process at the constant speed (in the above example, 5 mmHg/sec), a value approximated by a straight line determined by data of one point of the pressure Pc of the pressing cuff 23 and the depressurization speed may be used as the cuff pressure Pc. Alternatively, in the depressurization process, a value approximated by a straight line determined by data at two or more points of the pressure Pc of the pressing cuff 23 (that is, data of the pressure Pc at different times) may be used as the cuff pressure Pc. Alternatively, the temporary systolic blood pressure value SYS and the temporary diastolic blood pressure value DIA are once obtained by a general oscillometric method, and a value approximated by a straight line determined by the temporary systolic blood pressure value SYS and the temporary diastolic blood pressure value DIA may be used as the cuff pressure Pc.

(Fifth Modification)

In the above example, the blood pressure calculation is performed in the depressurization process, but the present invention is not limited to this configuration, and the blood pressure calculation may be performed in the pressurization process.

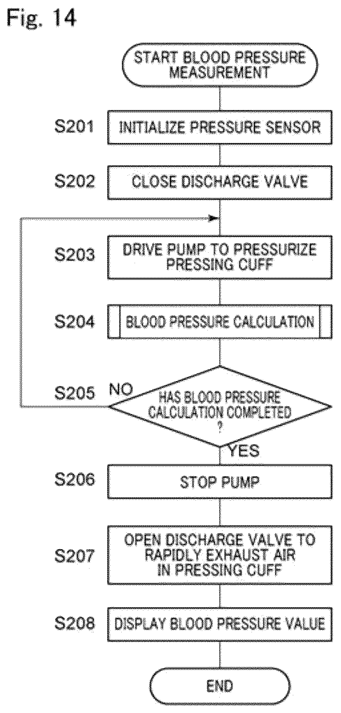

For example, FIG. 14 shows a schematic operation flow of blood pressure measurement by the sphygmomanometer 1 in FIG. 1, in which blood pressure calculation is performed in the pressurization process.

When the user (subject) turns on the measurement switch 52A provided on the main body 10 to instruct to start the measurement in the attached state (see FIG. 2) in which the cuff 20 is attached to the measurement target site (in this example, an upper arm) 90, the CPU 110 initializes the pressure sensor as shown in step S201 of FIG. 14. Specific processing of the initialization is the same as that described with respect to step S1 in FIG. 3.

In this example, as described with respect to the operation flow of FIG. 3, it is assumed that air as a pressure transmission fluid is sealed in a predetermined amount in the sensing cuff 21 (and the first air pipe 37) at the manufacturing stage of the sphygmomanometer 1.

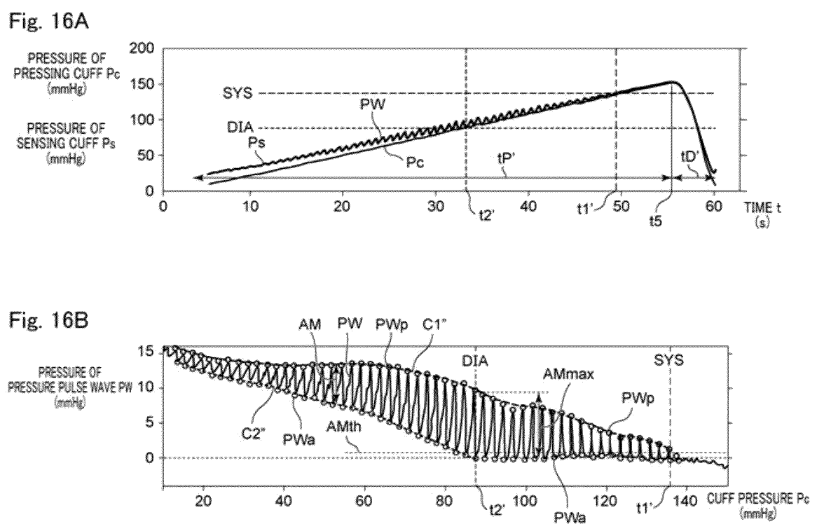

Next, in step S202 of FIG. 14, the CPU 110 closes the discharge valve 33 via the valve drive circuit 330. Subsequently, in step S203, the CPU 110 acts as a pressure control unit, drives the pump 32 via the pump drive circuit 320, and starts pressurization of the cuff 20 (to be precise, the pressing cuff 23) (pressurization process). While supplying air from the pump 32 to the pressing cuff 23 through the second air pipe 38, the CPU 110 controls the pressurization speed to be substantially constant (in this example, 3 mmHg/sec) from a value sufficiently lower than the expected diastolic blood pressure value DIA of the subject (in this example, about 10 mmHg) based on the output of the second pressure sensor 31. This causes the pressure Pc of the pressing cuff 23 to increase substantially linearly as indicated by a pressurization period tP′ in FIG. 16A. Since the sensing cuff 21 is pressed by the pressing cuff 23 toward the measurement target site 90 via the back plate 22, the pressure Ps of the sensing cuff 21 also increases with the pressure Pc of the pressing cuff 23.

In the pressurization process, as shown in step S204 of FIG. 14, the CPU 110 serves as a blood pressure calculation unit, and attempts to calculate the blood pressure value (the systolic blood pressure value SYS and the diastolic blood pressure value DIA) based on the data representing the pressure Ps of the sensing cuff 21 through the first pressure sensor 30 and the data representing the pressure Pc of the pressing cuff 23 through the second pressure sensor 31 acquired by this time. That is, the CPU 110 subtracts the direct current component of the data representing the pressure Pc of the pressing cuff 23 (which may be extracted from raw data via a low-pass filter) from the data representing the pressure Ps of the sensing cuff 21 in the pressurization process while associating the times of generation of the data with each other, and obtains the pressure at a rising start point PWa and the pressure at a peak point PWp indicated by the pressure pulse wave PW for each beat as shown in FIG. 16B. Then, as shown in FIG. 1613, in the pressurization process, the CPU 110 obtains, as the diastolic blood pressure value DIA, the pressure Pc of the pressing cuff 23 at the second time point t2′ at which the pressure at the rising start point PWa for each beat falls from the positive value to the zero level, and obtains, as the systolic blood pressure value SYS, the pressure Pc of the pressing cuff 23 at the first time point t1′ at which the pressure at the peak point PWp for each beat falls from the positive value to the zero level. The principle of the blood pressure calculation in the pressurization process is the same as that in the depressurization process described above except that the change in the pressure Pc of the pressing cuff 23 with the lapse of time is opposite to that in the depressurization process. A more specific way of calculating blood pressure will be described in detail later.

At this time point, in a case where the blood pressure value cannot be calculated yet due to a shortage of data (NO in step S205 in FIG. 14), the processing of steps S203 to S205 is repeated.

When the blood pressure value can be calculated in this way (YES in step S205), the CPU 110 acts as a pressure control unit, stops the pump 32 via the pump drive circuit 320 in step S206, and opens the discharge valve 33 via the valve drive circuit 330 to perform control to rapidly exhaust the air in the pressing cuff 23 in step S207. In FIG. 16A, a time point t5 when the pump 32 is stopped and a rapid exhaust period tD′ following the time point t5 are shown. Further, in step S208, the CPU 110 performs control to display the measurement result of the blood pressure value on the display 50 and store the measurement result of the blood pressure value in the memory 51.

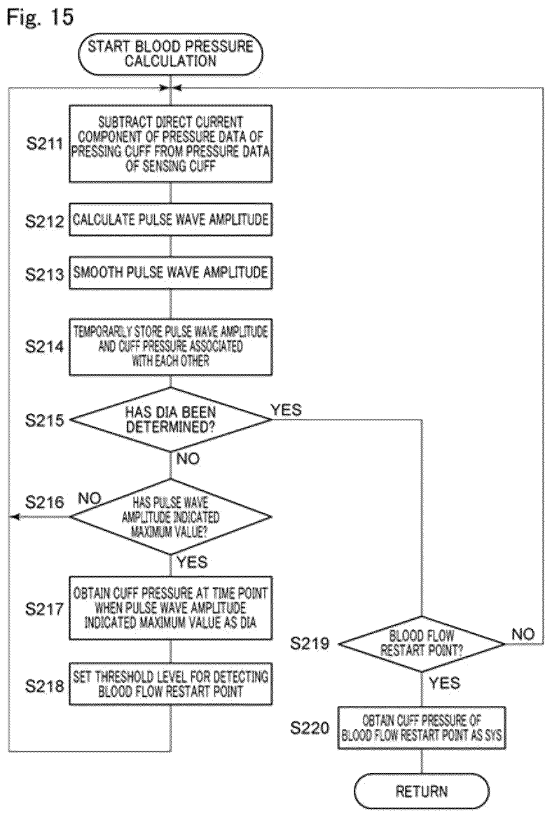

FIG. 15 shows a detailed flow of how to calculate the blood pressure in step S204 of FIG. 14.

In step S211 of FIG. 15, the CPU 110 subtracts the direct current component (cuff pressure Pc) of the pressure Pc of the pressing cuff 23 through the second pressure sensor 31 from the data representing the pressure Ps of the sensing cuff 21 through the first pressure sensor 30 while associating the times of generation of the data with each other, and obtains the pressure at the rising start point PWa and the pressure at the peak point PWp indicated by the pressure pulse wave PW for each beat as shown in FIG. 16B. In this example, a first curve C1″ connecting the rising start point PWa a second curve C2″ connecting the peak point PWp are shown in FIG. 16B.

Next, in step S212 of FIG. 15, the CPU 110 calculates a difference obtained by subtracting the pressure at the rising start point PWa from the pressure at the peak point PWp for each beat as the pulse wave amplitude AM shown in FIG. 16B.

Next, in step S213 of FIG. 15, in order to accurately obtain the systolic blood pressure value SYS and the diastolic blood pressure value DIA, the CPU 110 takes a moving average over a plurality of predetermined beats (five beats in this example) and smooths the pulse wave amplitude AM for each beat. Then, in step S214, the CPU 110 stores the smoothed data of each pulse wave amplitude AM in the memory 51 in time series in association with the data representing the cuff pressure Pc at the time point when the pulse wave amplitude AM is indicated. In this example, the cuff pressure Pc is extracted as a direct current component from raw data representing the pressure Pc of the pressing cuff 23 via a low-pass filter.

Next, in step S215 of FIG. 15, the CPU 110 determines whether the diastolic blood pressure value DIA has been determined. In the first turn of this processing, it is assumed that the diastolic blood pressure value DIA has not been determined yet (NO in step S215 in FIG. 15). In this case, the processing proceeds to step S216, and the CPU 110 refers to the contents stored in the memory 51 and determines whether the pulse wave amplitude AM has indicated a maximum value AMmax shown in FIG. 16B. At this time point, when the pulse wave amplitude AM has not yet indicated the maximum value AMmax (NO in step S216 in FIG. 15), the processing of steps S211 to S216 is repeated. On the other hand, when the pulse wave amplitude AM has indicated the maximum value AMmax (YES in step S216 in FIG. 15), the process proceeds to step S217, and the CPU 110 determines the time point at which the pulse wave amplitude AM indicated the maximum value AMmax as the second time point t2′ (that is, the second curve C2″ shown in FIG. 16B falls from the positive value to the zero level), and obtains the cuff pressure Pc corresponding to the determined second time point t2′ as the diastolic blood pressure value DIA. Here, whether the pulse wave amplitude AM has indicated the maximum value AMmax is determined by comparison between the pulse wave amplitudes AM, and thus can be determined with higher accuracy as compared with the case of comparison with a zero level (buried in noise). Thus, in the sphygmomanometer 1, the second time point t2′ can be accurately determined, whereby the diastolic blood pressure value DIA can be accurately obtained.

Next, in step S218 of FIG. 15, the CPU 110 multiplies the maximum value AMmax of the pulse wave amplitude AM by a predetermined ratio, and sets the threshold level AMth for determining the first time point t1′ (that is, when the first curve C1″ shown in FIG. 16B falls from the positive value to the zero level) as shown in FIG. 16B. Here, the “predetermined ratio” is set to exceed the actual noise level and be as small as possible in order to accurately determine the first time point t1′. The “predetermined ratio” is set to, for example, about 0.1 with respect to the maximum value AMmax of the pulse wave amplitude AM.

Subsequently, in step S219 in FIG. 15, the CPU 110 refers to the contents stored in the memory 51, and determines a time point at which the pulse wave amplitude AM crosses the threshold level AMth (indicating the blood flow restart point) in the pressurization process as the first time point W. More specifically, among the data in which the pulse wave amplitudes AM are lower than the threshold level AMth after the second time point t2′, the data in which the corresponding cuff pressure Pc is the minimum is searched, and the time point indicated by the data is determined as the first time point t1′. Then, in step S220 of FIG. 15, the CPU 110 obtains the cuff pressure Pc corresponding to the determined first time point t1′ as the systolic blood pressure value SYS. Here, whether the pulse wave amplitude AM exceeds the threshold level AMth is determined by comparison between the pulse wave amplitudes AM and the set threshold level AMth, and thus can be determined with higher accuracy as compared with the case of comparison with a zero level (buried in noise). Thus, in the sphygmomanometer 1, the first time point t1′ can be accurately determined, whereby the systolic blood pressure value SYS can be accurately obtained.

In this way, according to the sphygmomanometer 1, the blood pressure at the measurement target site 90 can be correctly measured in principle in a noninvasive manner not only in the depressurization process but also in the pressurization process. In the same way, according to the sphygmomanometer 1A of FIG. 11 as well, the blood pressure at the measurement target site 90 can be correctly measured in principle in a noninvasive manner not only in the depressurization process but also in the pressurization process.

(Other Modification)

In the above example, the planar shape of the sensing cuff 21 is a rounded rectangle, but the present invention is not limited to this configuration. The planar shape of the sensing cuff 21 may be, for example, a circular shape. As a result, only the volume change of the artery 91 at the place where the pressure Pc of the pressing cuff 23 is sufficiently applied to the pressure Ps of the sensing cuff 21 is further reflected. Thus, the measurement accuracy of the blood pressure is improved.

In the above example, the measurement target site 90 is an upper arm, and accordingly, the cuff 20 is a cuff for an upper arm, but the present invention is not limited to this configuration. The measurement target site 90 may be a wrist or a lower limb. Accordingly, the cuff 20 may be a cuff for a wrist or a cuff for a lower limb. In the example of the cuff for a wrist, the planar dimensions of the sensing cuff 21 are set to, for example, 2 cm×2 cm or less since the planar dimensions of the pressing cuff 23 are set smaller than that in the above example (example of the cuff for an upper arm).

In the above example, the “fluid” for pressurization and pressure transmission is air, but the present invention is not limited to this configuration. The “fluid” for pressurization and pressure transmission may be another gas or liquid. In particular, when the pressure transmitting “fluid” is sealed in the sensing cuff 21 at the manufacturing stage, the pressure transmitting “fluid” may be a liquid.

As described above, an electronic sphygmomanometer of the present disclosure is an electronic sphygmomanometer that noninvasively measures blood pressure at a measurement target site, the electronic sphygmomanometer comprising:
a pressing cuff having a bag shape configured to be attached around the measurement target site along a circumferential direction of the measurement target site to receive supply of a pressurization fluid and compress the measurement target site;
a sensing cuff having a bag shape disposed in a portion facing an artery of the measurement target site on an inner circumferential side of the pressing cuff, the sensing cuff containing a pressure transmission fluid separately from the pressing cuff, the sensing cuff configured to compress the artery of the measurement target site via the pressure transmission fluid by using a pressure of the pressing cuff and receive a pressure pulse wave from the artery;
a first pressure sensor that detects a pressure of the sensing cuff;
a second pressure sensor that detects the pressure of the pressing cuff;
a pressure control unit that controls the pressure of the pressing cuff by supplying the pressurization fluid to the pressing cuff or discharging the pressurization fluid from the pressing cuff; and
a blood pressure calculation unit that calculates a blood pressure value based on data representing the pressure of the sensing cuff from the first pressure sensor and data representing the pressure of the pressing cuff from the second pressure sensor in a process of changing the pressure of the pressing cuff with the pressure control unit,
wherein the blood pressure calculation unit
obtains a pressure at a rising start point and a pressure at a peak point indicated by the pressure pulse wave for each beat by subtracting a direct current component of data representing the pressure of the pressing cuff or a value approximate to the direct current component from data representing the pressure of the sensing cuff in the process of changing the pressure; and
obtains a first time point at which the pressure at the peak point for each beat transitions between a zero level and a positive value in the process of changing the pressure, obtains the pressure of the pressing cuff at the first time point as a systolic blood pressure value, obtains a second time point at which the pressure at the rising start point for each beat transitions between the zero level and the positive value, and obtains the pressure of the pressing cuff at the second time point as a diastolic blood pressure value.

In the present specification, the “pressure transmission fluid” may be enclosed in the sensing cuff at the manufacturing stage of the electronic sphygmomanometer, or may be contained in the sensing cuff and discharged from the sensing cuff every time the blood pressure is measured.

The “fluid” for pressurization fluid and pressure transmission fluid is typically air, but it may be another gas or liquid.

The “direct current component” of the data representing the pressure of the pressing cuff means a component obtained by removing a fluctuation component (for example, a fluctuation component for each beat derived from a pressure pulse wave from the artery) from the data representing the pressure of the pressing cuff. The “value approximate to the direct current component” refers to, for example, in a depressurization process at a constant depressurization speed, a value approximate to a straight line determined by data of one point of the pressure of the pressing cuff and the depressurization speed.

The “obtains a pressure at a rising start point and a pressure at a peak point indicated by the pressure pulse wave for each beat” means to obtain the pressure at the rising start point and the pressure at the peak point when the pressure pulse wave detected by the first pressure sensor indicates the rising start point and the peak point for each beat. For example, in a pressure section in which the pressure of the pressing cuff is higher than the systolic blood pressure value in the depressurization process, the blood flow in the artery is stopped, and thus, the “rising start point” and the “peak point” are not observed, and therefore, these pressures are not obtained.

The “first time point” and the “second time point” are names for convenience of distinguishing these time points from each other, and do not necessarily mean the order of these time points.

In the electronic sphygmomanometer of the present disclosure, a bag-shaped pressing cuff is attached around the measurement target site along a circumferential direction. In this attached state, separately from the pressing cuff, a bag-shaped sensing cuff is disposed in a portion of the measurement target site facing the artery on the inner circumferential side of the pressing cuff. For example, it is assumed that a pressure transmission fluid is sealed in the sensing cuff in advance at the manufacturing stage of the electronic sphygmomanometer.

During blood pressure measurement, the pressure control unit controls the pressure of the pressing cuff by supplying the pressurization fluid to the pressing cuff or discharging the pressurizing fluid from the pressing cuff. The sensing cuff compresses the artery of the measurement target site via the pressure transmission fluid with the pressure of the pressing cuff, and receives a pressure pulse wave from the artery. The first pressure sensor detects the pressure of the sensing cuff, and the second pressure sensor detects the pressure of the pressing cuff. The blood pressure calculation unit calculates blood pressure values (systolic blood pressure and diastolic blood pressure) based on data representing the pressure of the sensing cuff from the first pressure sensor and data representing the pressure of the pressing cuff from the second pressure sensor in a process of changing the pressure of the pressing cuff by the pressure control unit. Specifically, the blood pressure calculation unit subtracts a direct current component of the data representing the pressure of the pressing cuff or a value approximate to the direct current component from the data representing the pressure of the sensing cuff in the process of changing the pressure to obtain the pressure at the rising start point and the pressure at the peak point indicated by the pressure pulse wave for each beat. In addition, the blood pressure calculation unit obtains a first time point at which the pressure at the peak point for each beat transitions between a zero level and a positive value in the process of changing the pressure, obtains the pressure of the pressing cuff at the first time point as a systolic blood pressure value, obtains a second time point at which the pressure at the rise start point for each beat transitions between a zero level and a positive value, and obtains the pressure of the pressing cuff at the second time point as a diastolic blood pressure value.

This enables the electronic sphygmomanometer to correctly measure the blood pressure at the measurement target site in principle in a noninvasive manner. The reason for this will be described in detail in the embodiment section with reference to the drawings.

In the electronic sphygmomanometer of one embodiment,
the process of changing the pressure is a depressurization process after a blood flow through the artery was temporarily stopped by pressurization of the pressing cuff,
the first time point is a time point at which the pressure at the peak point for each beat starts to show the positive value from the zero level, and
the second time point is a time point at which the pressure at the rising start point for each beat starts to show the positive value from the zero level after the first time point.

In the electronic sphygmomanometer of this one embodiment, the first time point corresponds to a time point at which the blood flow has just resumed after the blood flow through the artery was temporarily stopped by pressurization of the pressing cuff, that is, a time point at which the pressure of the pressing cuff has just fallen below the systolic blood pressure. Thus, the pressure of the pressing cuff at the first time point is obtained as a systolic blood pressure value. The second time point corresponds to a time point at which the pressure of the pressing cuff has just fallen below the diastolic blood pressure. Thus, the pressure of the pressing cuff at the second time point is obtained as a diastolic blood pressure value.

In the electronic sphygmomanometer of one embodiment,
the process of changing the pressure is a pressurization process of the pressing cuff,
the second time point is a time point at which the pressure at the rising start point for each beat falls from the positive value to the zero level, and
the first time point is a time point at which the pressure at the peak point for each beat falls from the positive value to the zero level after the second time point.

In the electronic sphygmomanometer of this one embodiment, the second time point corresponds to a time point at which the pressure of the pressing cuff has just exceeded the diastolic blood pressure. Thus, the pressure of the pressing cuff at the second time point is obtained as a diastolic blood pressure value. The first time point corresponds to a time point at which the pressure of the pressing cuff has just exceeded the systolic blood pressure. Thus, the pressure of the pressing cuff at the first time point is obtained as a systolic blood pressure value.

In the electronic sphygmomanometer of one embodiment,
the sensing cuff is connected to the first pressure sensor via a first fluid pipe in a fluid communicable manner,
the pressing cuff is connected to the second pressure sensor via a second fluid pipe in a fluid communicable manner, and
the first fluid pipe and the second fluid pipe are separated from each other.

In the electronic sphygmomanometer of this one embodiment, the first fluid pipe and the second fluid pipe are separated from each other. Thus, for example, it is possible to prevent mixing of a fluctuation component (caused by a pressure pulse wave from the artery, for example) included in the pressure of the first fluid pipe connected to the sensing cuff as noise into the pressure of the second fluid pipe connected to the pressing cuff. Conversely, it is possible to prevent mixing of the fluctuation component included in the pressure of the second fluid pipe connected to the pressing cuff as noise into the pressure of the first fluid pipe connected to the sensing cuff.

In the electronic sphygmomanometer of one embodiment,
with regard to a width direction of the pressing cuff along a direction in which the artery of the measurement target site passes, a range occupied by the sensing cuff is within a remaining range excluding a predetermined range from an upstream side of the artery, of a width direction dimension of the pressing cuff.

It is assumed that, with regard to a width direction of the pressing cuff along the direction in which the artery of the measurement target site passes, the range occupied by the sensing cuff is, for example, within a range of ⅓ from the upstream side of the artery in the width direction dimension of the pressing cuff. Then, even when the pressure of the pressing cuff is in the pressure section higher than the systolic blood pressure value in the depressurization process and thus the blood flow in the artery is stopped, a pulse wave dirceting from an upstream side of the artery to immediately below the pressing cuff may be mixed as noise (fluctuation component) into the pressure of the sensing cuff detected by the first pressure sensor. Thus, in the electronic sphygmomanometer according to this one embodiment, with regard to the width direction of the pressing cuff along the direction in which the artery of the measurement target site passes, the range occupied by the sensing cuff is within the remaining range excluding the predetermined range from the upstream side of the artery, of the width direction dimension of the pressing cuff. Thus, when the pressure of the pressing cuff is in the pressure section higher than the systolic blood pressure value in the depressurization process and thus the blood flow in the artery is stopped, it is possible to prevent mixing of a pulse wave dirceting from the upstream side of the artery to immediately below the pressing cuff as noise (fluctuation component) into the pressure of the sensing cuff detected by the first pressure sensor.

The electronic sphygmomanometer of one embodiment further comprises a back plate interposed between the pressing cuff and the sensing cuff, wherein the back plate is configured to transmit the pressure of the pressing cuff to the sensing cuff and block transmission of a pressure fluctuation component between the pressing cuff and the sensing cuff.

In the electronic sphygmomanometer of this one embodiment, the pressure of the pressing cuff can be reliably transmitted to the sensing cuff with the back plate. Thus, the sensing cuff can reliably compress the artery of the measurement target site via the pressure transmission fluid with the pressure of the pressing cuff. At the same time, the back plate blocks transmission of a pressure fluctuation component between the pressing cuff and the sensing cuff. Thus, for example, it is possible to prevent mixing of a fluctuation component (caused by a pressure pulse wave from the artery, for example) included in the pressure of the sensing cuff as noise into the pressure of the pressing cuff. Conversely, it is possible to prevent mixing of the fluctuation component included in the pressure of the pressing cuff as noise into the pressure of the sensing cuff.

In the electronic sphygmomanometer of one embodiment,
the first fluid pipe is configured to be drawn out of a region in which the pressing cuff surrounds the measurement target site in a manner that the first fluid pipe is deviated from the artery in a planar view viewed from a thickness direction perpendicular to an outer circumferential surface of the measurement target site.

The “in a manner that the first fluid pipe is deviated from the artery” means being separated from the artery and not overlapping with the artery.

In the electronic sphygmomanometer according to this one embodiment, the first fluid pipe is drawn out of the region in which the pressing cuff surrounds the measurement target site in a manner that the first fluid pipe is deviated from the artery in a planar view viewed from a thickness direction perpendicular to the outer circumferential surface of the measurement target site. Thus, the first fluid pipe does not prevent the pressure of the pressing cuff from compressing the artery of the measurement target site via the sensing cuff. Further, the first fluid pipe does not prevent the sensing cuff from receiving the pressure pulse wave from the artery.

In the electronic sphygmomanometer of one embodiment,
the pressing cuff includes a through hole penetrating a portion of the pressing cuff in a thickness direction, the portion being in a region facing the sensing cuff, and
the first fluid pipe is drawn out from the sensing cuff to an outer circumferential side of the pressing cuff through the through hole of the pressing cuff.

The “thickness direction” of the pressing cuff (and the sensing cull) refers to a direction perpendicular to the outer circumferential surface of the measurement target site with the cuff attached to the measurement target site.

In the electronic sphygmomanometer according to this one embodiment, the first fluid pipe is drawn out from the sensing cuff to the outside of the pressing cuff through the through hole of the pressing cuff. Thus, the first fluid pipe does not prevent the pressure of the pressing cuff from compressing the artery of the measurement target site via the sensing cuff. Further, the first fluid pipe does not prevent the sensing cuff from receiving the pressure pulse wave from the artery.

In the electronic sphygmomanometer of one embodiment, a predetermined amount of the pressure transmission fluid is sealed in the sensing cuff.

The “predetermined amount” refers to, for example, an amount that can avoid a situation in which the bag forming the sensing cuff is crushed in the thickness direction by the pressure of the pressing cuff and the sheet forming the bag is brought into close contact. Avoiding such a situation enables the sensing cuff to reliably compress the artery of the measurement target site via the pressure transmission fluid with the pressure of the pressing cuff, and to reliably receive the pressure pulse wave from the artery.

In the electronic sphygmomanometer of this one embodiment, a predetermined amount of the pressure transmission fluid is sealed in the sensing cuff. Thus, for example, it is possible to omit time and effort for enclosing the pressure transmission fluid in the sensing cuff every time the blood pressure is measured.

The electronic sphygmomanometer of one embodiment further comprises a fluid containment control unit that performs control to supply the pressure transmission fluid to the sensing cuff via the first fluid pipe and enclose the pressure transmission fluid in advance of a start of pressurization of the pressing cuff with the pressure control unit and discharge the pressure transmission fluid from the sensing cuff via the first fluid pipe after a completion of calculation of the blood pressure value with the blood pressure calculation unit every time the blood pressure is measured.

In the electronic sphygmomanometer of this one embodiment, the pressure transmission fluid can be automatically sealed in the sensing cuff in advance of the start of pressurization of the pressing cuff every time the blood pressure is measured. Further, the pressure transmission fluid can be automatically discharged from the sensing cuff after the completion of the calculation of the blood pressure value.

In the electronic sphygmomanometer of one embodiment,
the pressure control unit includes a pump for supplying air as the pressurization fluid to the pressing cuff via the second fluid pipe and a discharge valve for discharging the air from the pressing cuff via the second fluid pipe,
the fluid containment control unit includes a switching valve connected between the first fluid pipe and the second fluid pipe, the switching valve being configured to be able to take a first position where the first fluid pipe and the second fluid pipe are separated from each other, and a second position where the first fluid pipe and the second fluid pipe are in a fluid communicable state and the pressing cuff is sealed, and
the fluid containment control unit is configured to:
supply air as the pressure transmission fluid to the sensing cuff by using the pump by switching the switching valve to the second position when supplying the pressure transmission fluid to the sensing cuff to enclose the pressure transmission fluid in advance of the start of pressurization of the pressing cuff with the pressure control unit;
maintain the switching valve at the first position during pressurization or depressurization of the pressing cuff with the pressure control unit; and
discharge the air from the sensing cuff by using the discharge valve by switching the switching valve to the second position when discharging the pressure transmission fluid from the sensing cuff after the completion of calculation of the blood pressure value with the blood pressure calculation unit.

In the electronic sphygmomanometer of this one embodiment, air can be automatically sealed in the sensing cuff as the pressure transmission fluid, and the air can be automatically discharged from the sensing cuff, with a relatively small number of components.

In the electronic sphygmomanometer according to one embodiment,
a storage unit capable of storing data is provided,
wherein the blood pressure calculation unit is configured to:
calculate, as a pulse wave amplitude, a difference obtained by subtracting the pressure at the rising start point from the pressure at the peak point for each beat, and store data of each pulse wave amplitude in the storage unit in time series in association with the data of the pressure of the pressing cuff at the time point when the pulse wave amplitude is indicated; and
determine, as the second time point, a time point at which the pulse wave amplitude showed a maximum value in the process of changing the pressure with reference to contents stored in the storage unit.

Normally, since the pressure pulse wave received by the sensing cuff has noise, it may be difficult to determine a time point (second time point) at which the pressure at the rising start point transitions between a zero level (buried in noise) and a positive value. Thus, in the electronic sphygmomanometer according to this one embodiment, the blood pressure calculation unit calculates, as a pulse wave amplitude, a difference obtained by subtracting the pressure at the rising start point from the pressure at the peak point for each beat, and stores data of each pulse wave amplitude in the process of changing the pressure in the storage unit in time series in association with the data of the pressure of the pressing cuff at the time point when the pulse wave amplitude is indicated. Then, the blood pressure calculation unit refers to contents stored in the storage unit and determines a time point at which the pulse wave amplitude showed the maximum value in the process of changing the pressure as the second time point. As a result, the pressure of the pressing cuff corresponding to the determined second time point is obtained as the diastolic blood pressure value. Here, whether the pulse wave amplitude indicates the maximum value is determined by comparison between the pulse wave amplitudes, and thus can be determined with higher accuracy as compared with the case of comparison with a zero level (buried in noise). Thus, in the electronic sphygmomanometer of this one embodiment, the second time point can be accurately determined, whereby the diastolic blood pressure value can be accurately obtained.

In the electronic sphygmomanometer according to an embodiment,
the blood pressure calculation unit is configured to:
set a threshold level for determining the first time point by multiplying the maximum value of the pulse wave amplitude by a predetermined ratio; and
determine a time point at which the pulse wave amplitude crosses the threshold level in the process of changing the pressure as the first time point with reference to contents stored in the storage unit.

The “predetermined ratio” is set to exceed the actual noise level and be as small as possible in order to accurately determine the first time point. The “predetermined ratio” with respect to the maximum value of the pulse wave amplitude is set to, for example, about 0.1.

Normally, since the pressure pulse wave received by the sensing cuff has noise, it may be difficult to determine a time point (first time point) at which the pressure at the peak point transitions between a zero level (buried in noise) and a positive value. Thus, in the electronic sphygmomanometer according to this one embodiment, the blood pressure calculation unit sets a threshold level for determining the first time point by multiplying the maximum value of the pulse wave amplitude by a predetermined ratio. Then, the blood pressure calculation unit refers to the contents stored in the storage unit and determines a time point at which the pulse wave amplitude crosses the threshold level in the process of changing the pressure as the first time point. As a result, the pressure of the pressing cuff corresponding to the determined first time point is obtained as the systolic blood pressure value. Here, since whether the pulse wave amplitude has crossed the threshold level is determined by comparing the pulse wave amplitude with the set threshold level, it can be determined with high accuracy as compared with the case of comparison with a zero level (buried in noise). Thus, in the electronic sphygmomanometer of this one embodiment, the first time point can be accurately determined, whereby the systolic blood pressure value can be accurately obtained.

In the electronic sphygmomanometer according to this one embodiment, the blood pressure calculation unit calculates the pulse wave amplitude for each beat, takes a moving average over a plurality of predetermined beats to smooth the pulse wave amplitude, and causes the storage unit to store in time series the smoothed pulse wave amplitude.

The “plurality of predetermined beats” is, for example, five beats.

Normally, since the blood pressure fluctuates for each beat, the pulse wave amplitude also fluctuates under the influence of the fluctuation. Thus, in the electronic sphygmomanometer according to this one embodiment, the blood pressure calculation unit calculates the pulse wave amplitude for each beat, then takes a moving average over a plurality of beats to smooth the pulse wave amplitude for each beat, and causes the storage unit to store in time series the smoothed pulse wave amplitude. As a result, the smoothed pulse wave amplitude is stored in the storage unit in time series. With this configuration, the systolic blood pressure value and the diastolic blood pressure value can be accurately obtained.

In another aspect, a blood pressure measurement method of the present disclosure is a blood pressure measurement method for noninvasively measuring blood pressure at a measurement target site, wherein
a pressing cuff having a bag shape configured to be attached around the measurement target site along a circumferential direction of the measurement target site to receive supply of a pressurization fluid and compress the measurement target site;
a sensing cuff having a bag shape disposed in a portion facing an artery of the measurement target site on an inner circumferential side of the pressing cuff, the sensing cuff containing a pressure transmission fluid separately from the pressing cuff, the sensing cuff configured to compress the artery of the measurement target site via the pressure transmission fluid by using a pressure of the pressing cuff and receive a pressure pulse wave from the artery;
a first pressure sensor that detects a pressure of the sensing cuff;
a second pressure sensor that detects the pressure of the pressing cuff; and
a pressure control unit that controls the pressure of the pressing cuff by supplying the pressurization fluid to the pressing cuff or discharging the pressurization fluid from the pressing cuff are provided,
the blood pressure measurement method comprising:
acquiring data representing the pressure of the sensing cuff from the first pressure sensor and data representing the pressure of the pressing cuff from the second pressure sensor in a process of changing the pressure of the pressing cuff with the pressure control unit;
obtaining a pressure at a rising start point and a pressure at a peak point indicated by the pressure pulse wave for each beat by subtracting a direct current component of data representing the pressure of the pressing cuff or a value approximate to the direct current component from data representing the pressure of the sensing cuff in the process of changing the pressure; and
obtaining a first time point at which the pressure at the peak point for each beat transitions between a zero level and a positive value in the process of changing the pressure, obtaining the pressure of the pressing cuff at the first time point as a systolic blood pressure value, obtaining a second time point at which the pressure at the rising start point for each beat transitions between the zero level and the positive value, and obtaining the pressure of the pressing cuff at the second time point as a diastolic blood pressure value.

According to the blood pressure measurement method of the present disclosure, the blood pressure at the measurement target site can be correctly measured in principle in a noninvasive manner.

In still another aspect, the electronic sphygmomanometer of the present disclosure is an electronic sphygmomanometer that noninvasively measures blood pressure at a measurement target site, the electronic sphygmomanometer comprising:
a pressing cuff having a bag shape configured to be attached around the measurement target site along a circumferential direction of the measurement target site to receive supply of a pressurization fluid and compress the measurement target site;
a sensing cuff having a bag shape disposed in a portion facing an artery of the measurement target site on an inner circumferential side of the pressing cuff, the sensing cuff containing a pressure transmission fluid separately from the pressing cuff, the sensing cuff configured to compress the artery of the measurement target site via the pressure transmission fluid by using a pressure of the pressing cuff and receive a pressure pulse wave from the artery;
a first pressure sensor that detects a pressure of the sensing cuff;
a second pressure sensor that detects the pressure of the pressing cuff;
a pressure control unit that controls the pressure of the pressing cuff by supplying the pressurization fluid to the pressing cuff or discharging the pressurization fluid from the pressing cuff; and
a blood pressure calculation unit that calculates a blood pressure value based on data representing the pressure of the sensing cuff from the first pressure sensor and data representing the pressure of the pressing cuff from the second pressure sensor in a process of changing the pressure of the pressing cuff with the pressure control unit,
wherein the blood pressure calculation unit:
obtains a pressure at a rising start point and a pressure at a peak point indicated by the pressure pulse wave for each beat by subtracting a direct current component of data representing the pressure of the pressing cuff or a value approximate to the direct current component from data representing the pressure of the sensing cuff in the process of changing the pressure; and
obtains, on a coordinate plane defined by a horizontal axis representing time and a vertical axis representing pressure, a first time point corresponding to a point at which a first curve transitions between a zero level and a positive value, the first curve connecting the peak point for each beat in the process of changing the pressure, obtains the pressure of the pressing cuff at the first time point as a systolic blood pressure value, obtains, on the coordinate plane, a second time point corresponding to a point at which a second curve transitions between a zero level and a positive value, the second curve connecting the rising start point for each beat in the process of the changing the pressure.

In the present specification, “obtains, on a coordinate plane . . . a first time point corresponding to a point at which a first curve transitions between a zero level and a positive value” means that it is sufficient to obtain the first time point, and it is not always necessary to perform the process of drawing the first curve on the coordinate plane. In the same way, “obtains, on the coordinate plane . . . a second time point corresponding to a point at which a second curve transitions between a zero level and a positive value” means that it is sufficient to obtain the second time point, and it is not always necessary to perform the process of drawing the second curve on the coordinate plane.

The “first time point” and the “second time point” are names for convenience of distinguishing these time points from each other, and do not necessarily mean the order of these time points.

According to the electronic sphygmomanometer of the present disclosure, the blood pressure at the measurement target site can be correctly measured in principle in a noninvasive manner.

As is clear from the above, according to the electronic sphygmomanometer and the blood pressure measurement method of the present disclosure, the blood pressure at the measurement target site can be correctly measured in principle in a noninvasive manner.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. An electronic sphygmomanometer that noninvasively measures blood pressure at a measurement target site, the electronic sphygmomanometer comprising:
a pressing cuff having a bag shape configured to be attached around the measurement target site along a circumferential direction of the measurement target site to receive supply of a pressurization fluid and compress the measurement target site;
a sensing cuff having a bag shape disposed in a portion facing an artery of the measurement target site on an inner circumferential side of the pressing cuff, the sensing cuff containing a pressure transmission fluid separately from the pressing cuff, the sensing cuff configured to compress the artery of the measurement target site via the pressure transmission fluid by using a pressure of the pressing cuff and receive a pressure pulse wave from the artery;
a first pressure sensor that detects a pressure of the sensing cuff;
a second pressure sensor that detects the pressure of the pressing cuff;
a pressure control unit that controls the pressure of the pressing cuff by supplying the pressurization fluid to the pressing cuff or discharging the pressurization fluid from the pressing cuff; and
a blood pressure calculation unit that calculates a blood pressure value based on data representing the pressure of the sensing cuff from the first pressure sensor and data representing the pressure of the pressing cuff from the second pressure sensor in a process of changing the pressure of the pressing cuff with the pressure control unit,
wherein the blood pressure calculation unit
obtains a pressure at a rising start point and a pressure at a peak point indicated by the pressure pulse wave for each beat by subtracting a direct current component of data representing the pressure of the pressing cuff or a value approximate to the direct current component from data representing the pressure of the sensing cuff in the process of changing the pressure; and
obtains a first time point at which the pressure at the peak point for each beat transitions between a zero level and a positive value in the process of changing the pressure, obtains the pressure of the pressing cuff at the first time point as a systolic blood pressure value, obtains a second time point at which the pressure at the rising start point for each beat transitions between the zero level and the positive value, and obtains the pressure of the pressing cuff at the second time point as a diastolic blood pressure value.

2. The electronic sphygmomanometer according to claim 1, wherein
the process of changing the pressure is a depressurization process after a blood flow through the artery was temporarily stopped by pressurization of the pressing cuff,
the first time point is a time point at which the pressure at the peak point for each beat starts to show the positive value from the zero level, and
the second time point is a time point at which the pressure at the rising start point for each beat starts to show the positive value from the zero level after the first time point.

3. The electronic sphygmomanometer according to claim 1, wherein
the process of changing the pressure is a pressurization process of the pressing cuff,
the second time point is a time point at which the pressure at the rising start point for each beat falls from the positive value to the zero level, and
the first time point is a time point at which the pressure at the peak point for each beat falls from the positive value to the zero level after the second time point.

4. The electronic sphygmomanometer according to claim 1, wherein
the sensing cuff is connected to the first pressure sensor via a first fluid pipe in a fluid communicable manner,
the pressing cuff is connected to the second pressure sensor via a second fluid pipe in a fluid communicable manner, and
the first fluid pipe and the second fluid pipe are separated from each other.

5. The electronic sphygmomanometer according to claim 1 wherein
with regard to a width direction of the pressing cuff along a direction in which the artery of the measurement target site passes, a range occupied by the sensing cuff is within a remaining range excluding a predetermined range from an upstream side of the artery, of a dimension of the pressing cuff in the width direction.

6. The electronic sphygmomanometer according to claim 1, further comprising a back plate interposed between the pressing cuff and the sensing cuff wherein the back plate is configured to transmit the pressure of the pressing cuff to the sensing cuff and block transmission of a pressure fluctuation component between the pressing cuff and the sensing cuff.

7. The electronic sphygmomanometer according to claim 4, wherein
the first fluid pipe is configured to be drawn out of a region in which the pressing cuff surrounds the measurement target site in a manner that the first fluid pipe is deviated from the artery in a planar view viewed from a thickness direction perpendicular to an outer circumferential surface of the measurement target site.

8. The electronic sphygmomanometer according to claim 4, wherein
the pressing cuff includes a through hole penetrating a portion of the pressing cuff in a thickness direction, the portion being in a region facing the sensing cuff, and
the first fluid pipe is drawn out from the sensing cuff to an outer circumferential side of the pressing cuff through the through hole of the pressing cuff.

9. The electronic sphygmomanometer according to claim 1, wherein
a predetermined amount of the pressure transmission fluid is sealed in the sensing cuff.

10. The electronic sphygmomanometer according to claim 4, the electronic sphygmomanometer further comprising a fluid containment control unit that performs control to supply the pressure transmission fluid to the sensing cuff via the first fluid pipe and enclose the pressure transmission fluid in advance of a start of pressurization of the pressing cuff with the pressure control unit and discharge the pressure transmission fluid from the sensing cuff via the first fluid pipe after a completion of calculation of the blood pressure value with the blood pressure calculation unit every time the blood pressure is measured.

11. The sphygmomanometer according to claim 10, wherein
the pressure control unit includes a pump for supplying air as the pressurization fluid to the pressing cuff via the second fluid pipe and a discharge valve for discharging the air from the pressing cuff via the second fluid pipe,
the fluid containment control unit includes a switching valve connected between the first fluid pipe and the second fluid pipe, the switching valve being configured to be able to take a first position where the first fluid pipe and the second fluid pipe are separated from each other, and a second position where the first fluid pipe and the second fluid pipe are in a fluid communicable state and the pressing cuff is sealed, and
the fluid containment control unit is configured to:
supply air as the pressure transmission fluid to the sensing cuff by using the pump by switching the switching valve to the second position when supplying the pressure transmission fluid to the sensing cuff to enclose the pressure transmission fluid in advance of the start of pressurization of the pressing cuff with the pressure control unit;
maintain the switching valve at the first position during pressurization or depressurization of the pressing cuff with the pressure control unit; and
discharge the air from the sensing cuff by using the discharge valve by switching the switching valve to the second position when discharging the pressure transmission fluid from the sensing cuff after the completion of calculation of the blood pressure value with the blood pressure calculation unit.

12. A blood pressure measurement method for noninvasively measuring blood pressure at a measurement target site, wherein
a pressing cuff having a bag shape configured to be attached around the measurement target site along a circumferential direction of the measurement target site to receive supply of a pressurization fluid and compress the measurement target site;
a sensing cuff having a bag shape disposed in a portion facing an artery of the measurement target site on an inner circumferential side of the pressing cuff, the sensing cuff containing a pressure transmission fluid separately from the pressing cuff, the sensing cuff configured to compress the artery of the measurement target site via the pressure transmission fluid by using a pressure of the pressing cuff and receive a pressure pulse wave from the artery;
a first pressure sensor that detects a pressure of the sensing cuff;
a second pressure sensor that detects the pressure of the pressing cuff; and
a pressure control unit that controls the pressure of the pressing cuff by supplying the pressurization fluid to the pressing cuff or discharging the pressurization fluid from the pressing cuff are provided,
the blood pressure measurement method comprising:
acquiring data representing the pressure of the sensing cuff from the first pressure sensor and data representing the pressure of the pressing cuff from the second pressure sensor in a process of changing the pressure of the pressing cuff with the pressure control unit;
obtaining a pressure at a rising start point and a pressure at a peak point indicated by the pressure pulse wave for each beat by subtracting a direct current component of data representing the pressure of the pressing cuff or a value approximate to the direct current component from data representing the pressure of the sensing cuff in the process of changing the pressure; and
obtaining a first time point at which the pressure at the peak point for each beat transitions between a zero level and a positive value in the process of changing the pressure, obtaining the pressure of the pressing cuff at the first time point as a systolic blood pressure value, obtaining a second time point at which the pressure at the rising start point for each beat transitions between the zero level and the positive value, and obtaining the pressure of the pressing cuff at the second time point as a diastolic blood pressure value.
